(19) United States
(12) Patent Application Publication
(10) Pub. No.: US 2025/0138028 A1
(43) Pub. Date: May 1, 2025

(54) Serum Immune-Based Biomarkers for Use in ALS Therapy

(71) Applicants: The Methodist Hospital, Houston, TX (US); Coya Therapeutics, Inc., Houston, TX (US)

(72) Inventors: Stanley Hersh Appel, Houston, TX (US); David Robert Beers, Houston, TX (US); Howard Berman, Houston, TX (US)

(21) Appl. No.: 18/692,235

(22) PCT Filed: Sep. 14, 2022
(86) PCT No.: PCT/US2022/076412

§ 371 Date: Mar. 14, 2024

Related U.S. Application Data
(60) Provisional application No. 63/244,995, filed on Sep. 16, 2021.

(51) Int. Cl. G01N 33/68; A61K 35/17; G01N 33/92

(52) U.S. Cl. CPC G01N 33/6896; A61K 35/17; G01N 33/6869; G01N 33/92; G01N 2333/54; G01N 2333/70596; G01N 2800/2835; G01N 2800/52

(57) ABSTRACT

The present disclosure provides methods for selecting a patient diagnosed with amyotrophic lateral sclerosis (ALS) for an ALS therapy, methods for assessing a ALS patient's likely responsiveness to an ALS therapy, methods for treating an ALS patient with an ALS therapy, methods for monitoring efficacy of an ALS therapy, and methods for assessing the likely progression of ALS in a patient, in which methods serum concentrations of one or more serum immune-based biomarkers are determined.

This application claims the benefit of U.S. Provisional Application No. 63/244,995 filed Sep. 16, 2021, the content of which is incorporated herein in its entirety.

### 1. FIELD

Provided herein are serum-immune based biomarkers and uses thereof in methods for selecting a patient diagnosed with amyotrophic lateral sclerosis (ALS) for an ALS therapy. Methods for treating an ALS patient, and for monitoring efficacy of treatment, are also provided herein.

### 2. BACKGROUND

Amyotrophic lateral sclerosis (ALS) is the most common form of adult neuromuscular disease, and is invariably fatal. ALS is considered a multifactorial, multisystem disease in which the CNS and peripheral immune systems both play important roles in the development and progression of disease.

Increasingly, studies point to immune system involvement in the progression of diseases such as ALS, and point to dysfunction of immune cells as a mediator of disease pathogenesis. The complex signaling mechanisms and built-in redundancies of the immune system and its constituents may help explain the ineffectiveness of single drug/single target anti-inflammatory approaches.

It has been demonstrated that in ALS patients regulatory T cells (Tregs) are progressively reduced in number and are less effective in promoting immune suppression. Recently, Treg cell therapy has emerged as a promising therapy for ALS, and may represent a more global approach to suppressing immune system dysfunction contributing to disease. For example, clinical trials involving administration of expanded autologous Tregs to ALS patients report that the Treg therapy slowed progression rates during early and later stages of the disease, and that Treg suppressive function correlated with the slowing of disease progression (Thonhoff et al., Neurol. Neuroimmunol. Neuroinflamm. 5 (4): e465 (2018)).

There exists a need for the development of ALS biomarkers that can accurately reflect patients' disease burdens, progression rates, and responsiveness to treatments such as Treg therapy. Such biomarkers could be clinically useful for prognosis of clinical course, prediction of treatment response, and determination of treatment efficacy.

### 3. SUMMARY

In one aspect, provided herein are methods for selecting a patient for amyotrophic lateral sclerosis (ALS) therapy.

In some embodiments, a method for selecting a patient for ALS therapy comprises: (a) determining a concentration of interleukin 17F (IL-17F) in a serum sample collected from a patient diagnosed with or suspected of having ALS, wherein if the IL-17F concentration in the serum sample is at least 2.0 pg/mL the patient is excluded from a treatment with the ALS therapy, otherwise the patient is selected for the treatment with the ALS therapy; and (b) administering the ALS therapy to the selected patient.

In certain embodiments, the ALS therapy comprises a T regulatory cell (Treg) infusion.

In certain embodiments, the ALS therapy comprises a plurality of Treg infusions.

In certain embodiments, the serum sample is collected from the patient prior to the ALS therapy.

In certain embodiments, the serum sample is collected from the patient prior to any Treg infusion.

In certain embodiments, the serum sample is collected from the patient after a Treg infusion has been administered to the patient.

In certain embodiments, the serum sample is collected from the patient on the day following the Treg infusion.

In one aspect, provided herein are methods of treating ALS in a patient.

In some embodiments, a method of treating ALS comprises: administering an ALS therapy to a patient diagnosed with ALS, wherein it has been determined that a serum sample collected from the patient contains an interleukin 17F (IL-17F) concentration of less than 2.0 pg/mL.

In certain embodiments, the ALS therapy comprises a Treg infusion.

In certain embodiments, the ALS therapy comprises a plurality of Treg infusions.

In certain embodiments, the serum sample had been collected from the patient prior to the ALS therapy.

In certain embodiments, the serum sample had been collected from the patient prior any Treg infusion.

In certain embodiments, the serum sample had been collected from the patient after a Treg infusion had been administered to the patient.

In certain embodiments, the serum sample had been collected from the patient on the day following the Treg infusion.

In some embodiments, a method for selecting a patient for ALS therapy comprises: (a) determining whether a concentration of at least one serum immune-based biomarker in a serum sample collected from a patient diagnosed with or suspected of having ALS is less than, equal to, or greater than, a reference concentration, wherein the at least one serum immune-based biomarker is IL-17F, oxidized low-density lipoprotein receptor 1 (OLR1), neurofilament light chain (NF-L), oxidized low-density lipoprotein (ox-LDL), or interleukin 17C (IL-17C); and wherein if the concentration of the at least one serum immune-based biomarker is greater than the reference concentration, the patient is excluded from a treatment with the ALS therapy, otherwise the patient is selected for the treatment with the ALS therapy; and (b) administering the ALS therapy to the selected patient.

In certain embodiments, the concentration of one serum immune-based biomarker is determined.

In certain embodiments, concentrations of at least two serum immune-based biomarkers are each determined to be less than, equal to, or greater than, a reference concentration, and if the concentrations of two serum immune-based biomarkers are each determined to be greater than its reference concentration, the patient is excluded from a treatment with the ALS therapy, otherwise the patient is selected for the treatment with the ALS therapy.

In certain embodiments, concentrations of at least three serum immune-based biomarkers are each determined to be less than, equal to, or greater than, a reference concentration, and if the concentrations of three serum immune-based biomarkers are each determined to be greater than its reference concentration, the patient is excluded from a treatment with the ALS therapy, otherwise the patient is selected for the treatment with the ALS therapy.

In certain embodiments, concentrations of at least four serum immune-based biomarkers are each determined to be less than, equal to, or greater than, a reference concentration, and if the concentrations of four serum immune-based biomarkers are each determined to be greater than its reference concentration, the patient is excluded from a treatment with the ALS therapy, otherwise the patient is selected for the treatment with the ALS therapy.

In certain embodiments, concentrations of at least five serum immune-based biomarkers are each determined to be less than, equal to, or greater than, a reference concentration, and if the concentrations of five serum immune-based biomarkers are each determined to be greater than its reference concentration, the patient is excluded from a treatment with the ALS therapy, otherwise the patient is selected for the treatment with the ALS therapy.

In certain embodiments, the ALS therapy comprises a Treg infusion.

In certain embodiments, the ALS therapy comprises a plurality of Treg infusions.

In certain embodiments, the serum sample is collected from the patient prior to the ALS therapy.

In certain embodiments, the serum sample is collected from the patient prior to any Treg infusion.

In certain embodiments, the serum sample is collected from the patient after a Treg infusion has been administered to the patient.

In certain embodiments, the serum sample is collected from the patient on the day following the Treg infusion.

In some embodiments, a method of treating ALS comprises: administering an ALS therapy to a patient diagnosed with ALS, wherein it has been determined that a serum sample collected from the patient contains a concentration of at least one serum immune-based biomarker that is less than, or equal to, a reference concentration, wherein the at least one serum immune-based biomarker is IL-17F, OLR1, NF-L, ox-LDL, or IL-17C.

In certain embodiments, a concentration of one serum immune-based biomarker has been determined to be less than, or equal to, a reference concentration.

In certain embodiments, a concentration of at least two serum immune-based biomarkers had been determined to be less than, or equal to, a reference concentration. In certain embodiments, a concentration of at least three serum immune-based biomarkers had been determined to be less than, or equal to, a reference concentration. In certain embodiments, a concentration of at least four serum immune-based biomarkers had been determined to be less than, or equal to, a reference concentration. In certain embodiments, a concentration of at least five serum immune-based biomarkers had been determined to be less than, or equal to, a reference concentration.

In certain embodiments, the ALS therapy comprises a Treg infusion.

In certain embodiments, the ALS therapy comprises a plurality of Treg infusions.

In certain embodiments, the serum sample had been collected from the patient prior to the ALS therapy.

In certain embodiments, the serum sample had been collected from the patient prior any Treg infusion.

In certain embodiments, the serum sample had been collected from the patient after a Treg infusion had been administered to the patient.

In certain embodiments, the serum sample had been collected from the patient on the day following the Treg infusion.

In certain embodiments, the reference concentration is obtained from healthy individuals.

In certain embodiments, the reference concentration is a concentration in a serum sample obtained from the patient prior to any Treg infusion being administered to the patient.

In some embodiments of the methods provided herein, a reference concentration for the at least one biomarker comprising ox-LDL, OLR1, NF-L, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| NF-L | 0.88 ± 0.21 | ng/ml |
| IL-17F | 0.34 ± 0.54 | pg/mL |
| IL-17C | 8.47 ± 3.08 | pg/mL |

In certain embodiments, the at least one serum immune-based biomarkers includes Ox-LDL.

In certain embodiments, the at least one serum immune-based biomarkers includes OLR1.

In certain embodiments, the at least one serum immune-based biomarkers includes NF-L.

In certain embodiments, the at least one serum immune-based biomarkers includes IL-17F.

In certain embodiments, the at least one serum immune-based biomarkers includes IL-17C.

In one aspect, provided herein are methods of monitoring efficacy of Treg therapy to treat ALS.

In some embodiments, a method of monitoring efficacy of a Treg therapy comprises: (a) administering a Treg therapy to a patient diagnosed with ALS, wherein the Treg therapy comprises a first Treg infusion; and (b) determining whether a concentration of at least one serum immune-based biomarker in a serum sample collected from the patient after the first Treg infusion is less than, equal to, or greater than, a reference concentration, wherein the at least one serum immune-based biomarker comprises: ox-LDL, OLR1, soluble CD14 (sCD14), lipopolysaccharide binding protein (LBP), C reactive protein (CRP), 4-hydroxynonenal (4-HNE), IL-17F, or IL-17C; wherein the ALS therapy has poor efficacy where the concentration of the at least one serum immune-based biomarker is greater than its reference concentration.

In certain embodiments, the method further comprises administering the Treg therapy comprising a second Treg infusion to the patient after steps (a) and (b) if the concentration of the at least one serum immune-based biomarker is equal to or less than its reference concentration in step (b).

In certain embodiments, Treg therapy comprising a plurality of Treg infusions is administered to the patient if the concentration of the at least one serum immune-based biomarker is equal to or less than its reference concentration in a serum sample collected from the patient after each Treg infusion of the plurality of Treg infusions.

In certain embodiments, the ALS therapy has poor efficacy where the concentration of one serum immune-based biomarker is greater than its reference concentration. In certain embodiments, the ALS therapy has poor efficacy where the concentration of at least 2 serum immune-based biomarkers are each greater than its reference concentration. In certain embodiments, the ALS therapy has poor efficacy where the concentration of at least 3 serum immune-based biomarkers are each greater than its reference concentration. In certain embodiments, the ALS therapy has poor efficacy where the concentration of at least 4 serum immune-based biomarkers are each greater than its reference concentration. In certain embodiments, the ALS therapy has poor efficacy where the concentration of at least 5 serum immune-based biomarkers are each greater than its reference concentration.

In some embodiments, a method of treating ALS comprises: (a) administering a first Treg infusion to a patient diagnosed with ALS; (b) comparing a concentration of at least one serum immune-based biomarker in a serum sample obtained from the patient after the first Treg infusion to a reference concentration, wherein the at least one immune-based biomarker comprises ox-LDL, OLR1, sCD14, LBP, CRP, 4-HNE, IL-17F, or IL-17C; and (c) administering an ALS therapy comprising a second Treg infusion to the patient if the concentration of the at least one serum immune-based biomarker is equal to or below the reference concentration.

In certain embodiments, the ALS therapy is administered to the patient if the concentration of one serum immune-based biomarker is equal to or below the reference concentration.

In certain embodiments, the ALS therapy is administered to the patient if the concentration of at least 2 serum immune-based biomarkers are each equal to or below its reference concentration. In certain embodiments, the ALS therapy is administered to the patient if the concentration of at least 3 serum immune-based biomarkers are each equal to or below its reference concentration. In certain embodiments, the ALS therapy is administered to the patient if the concentration of at least 4 serum immune-based biomarkers are each equal to or below its reference concentration. In certain embodiments, the ALS therapy is administered to the patient if the concentration of at least 5 serum immune-based biomarkers are each equal to or below its reference concentration.

In certain embodiments, the reference concentration for the at least one serum immune-based biomarker is obtained from healthy individuals.

In certain embodiments, the reference concentration for the at least one serum immune-based biomarker is a concentration in a serum sample obtained from the patient prior to any Treg infusion being administered to the patient.

In certain embodiments, the reference concentration for each of ox-LDL, OLR1, sCD14, LBP, CRP, 4-HNE, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| 4-HNE | 5 | ug/mL |
| IL-17F | 0.34 ± 0.54 | pg/mL |
| IL-17C | 8.47 ± 3.08 | pg/mL. |

In some embodiments, provided herein is a method for treating a patient with a Treg therapy wherein the patient is suffering from ALS.

In certain embodiments, the method comprises: (a) administering to the patient a Treg therapy comprising Treg infusions being administered to the patient on different days; (b) comparing concentrations of at least one serum immune-based biomarker in serum samples to a reference concentration, wherein each of the serum sample is obtained from the patient after a Treg infusion, wherein the at least one serum immune-based biomarker comprises ox-LDL, OLR1, sCD14, LBP, CRP, 4-HNE, IL-17F, or IL-17C; and (c) maintaining the patient on the Treg therapy comprising administering Treg infusions following step (b), if the concentration of the at least one serum immune-based biomarker is at, or less than, the reference concentration in at least one of serum samples.

In certain embodiments, the patient is maintained on the Treg therapy if the concentration of the at least one serum immune-based biomarker is at, or less than, the reference concentration in all or at least 50% of the serum samples.

In certain embodiments, the at least one serum immune-based biomarker consists of one serum immune-based biomarker.

In certain embodiments, the at least one serum immune-based biomarker comprises at least 2 serum immune-based biomarkers. In certain embodiments, the at least one serum immune-based biomarker comprises at least 3 serum immune-based biomarkers. In certain embodiments, the at least one serum immune-based biomarker comprises at least 4 serum immune-based biomarkers. In certain embodiments, the at least one serum immune-based biomarker comprises at least 5 serum immune-based biomarkers.

In certain embodiments, the reference concentration for the at least one serum immune-based biomarker is obtained from healthy individuals.

In certain embodiments, the reference concentration for the at least one serum immune-based biomarker is a concentration in a serum sample obtained from the patient prior to any Treg infusion being administered to the patient.

In certain embodiments, the reference concentration for each of ox-LDL, OLR1, sCD14, LBP, CRP, 4-HNE. IL-17F. or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| 4-HNE | 5 | ug/mL |
| IL-17F | 0.34 ± 0.54 | pg/mL |
| IL-17C | 8.47 ± 3.08 | pg/mL. |

In some embodiments, provided herein is a method for treating ALS in a patient diagnosed therewith, the method comprising: (a) determining whether the concentration of at least one serum immune-based biomarker in a serum sample obtained from a patient diagnosed with ALS is (i) elevated, or (ii) at, or below, a reference concentration, wherein the serum sample is obtained from the patient after being administered with a Treg infusion, wherein the at least one serum immune-based biomarker comprises ox-LDL, OLR1, sCD14, LBP, CRP, 4-HNE, IL-17F, or IL-17C; and (b) administering to the patient a Treg therapy comprising a plurality of Treg infusions if the concentration of the at least one serum immune-based biomarker is determined to be (ii) at, or below, its reference concentration.

In certain embodiments, the Treg therapy is administered to the patient if the concentration of one serum immune-based biomarker is determined to be (ii) at, or below, its reference concentration.

In certain embodiments, the Treg therapy is administered to the patient if the concentration of at least 2 serum immune-based biomarkers are each determined to be (ii) at, or below, its reference concentration. In certain embodiments, the Treg therapy is administered to the patient if the concentration of at least 3 serum immune-based biomarkers are each determined to be (ii) at, or below, its reference concentration. In certain embodiments, the Treg therapy is administered to the patient if the concentration of at least 4 serum immune-based biomarkers are each determined to be (ii) at, or below, its reference concentration. In certain embodiments, the Treg therapy is administered to the patient if the concentration of at least 5 serum immune-based biomarkers are each determined to be (ii) at, or below, its reference concentration.

In some embodiments of the methods provided, the reference concentration for the at least one serum immune-based biomarker is obtained from healthy individuals.

In other embodiments of the methods provided, the reference concentration for the at least one serum immune-based biomarker is a concentration in a serum sample obtained from the patient prior to any Treg infusion being administered to the patient.

In yet other embodiments of the methods provided, the reference concentration for the at least one serum immune-based biomarker comprising ox-LDL, OLR1, sCD14, LBP, CRP, 4-HNE, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| 4-HNE | 5 | ug/mL |
| IL-17F | 0.34 ± 0.54 | pg/mL |
| IL-17C | 8.47 ± 3.08 | pg/mL. |

In certain embodiments of the methods provided, the at least one serum immune-based biomarker comprises ox-LDL, OLR1, sCD14, or LBP. In certain embodiments, the at least one serum immune-based biomarker comprises ox-LDL, OLR1, IL-17C, or IL-17F. In certain embodiments, the at least one serum immune-based biomarker comprises ox-LDL. In certain embodiments, the at least one serum immune-based biomarker comprises OLR1. In certain embodiments, the at least one serum immune-based biomarker comprises sCD14. In certain embodiments, the at least one serum immune-based biomarker comprises IL-17C. In certain embodiments, the at least one serum immune-based biomarker comprises IL-17F.

In some embodiments of the methods provided the concentration of the at least one serum immune-based biomarker is determined using an enzyme-linked immunosorbent assay (ELISA).

In one aspect provided herein is a method for treating ALS in a patient diagnosed therewith, the method comprising: (a) determining whether the concentration of at least one serum immune-based biomarker in a serum sample obtained from a patient diagnosed with ALS is at or below that of a reference concentration of the at least one serum immune-based biomarker, wherein the serum sample is obtained from the patient after being administered with a first ALS therapy, wherein the at least one serum immune-based biomarker comprises ox-LDL, OLR1, sCD14, LBP, CRP, 4-HNE, IL-17F, or IL-17C; and (b) administering to the patient a second ALS therapy if the concentration of the at least one serum immune-based biomarker is determined to be at or below its reference concentration. In certain embodiments, the first ALS therapy is the same as the second ALS therapy. In certain embodiments, the first ALS therapy is different than the second ALS therapy.

In certain embodiments, the first and/or second ALS therapy comprises administering one or more Treg infusions to the patient.

In certain embodiments, the first and/or second ALS therapy comprises administering a CTLA-4 fusion protein and IL-2 to the patient. In some embodiments, the CTLA-4 fusion protein is abatacept. In certain embodiments, the IL-2 is aldesleukin.

In certain embodiments, the first and/or second ALS therapy comprises administering one or more Treg extracellular vesicle (EV), e.g., exosome, infusions to the patient.

In some embodiments, the at least one serum immune-based biomarker comprises ox-LDL or 4-HNE.

In one aspect, provided herein is a method for treating ALS in a patient diagnosed therewith, the method comprising: administering a first ALS therapy to the ALS patient; assessing the responsiveness of the ALS patient to the first ALS therapy; and continuing to administer the first ALS therapy to the ALS patient if the ALS patient is assessed to be responsive to the first ALS therapy, wherein the responsiveness of the ALS patient to the ALS therapy comprises comparing the serum concentration of at least one immune-based biomarker to a reference concentration of the of least one immune-based biomarker, wherein the ALS patient is assessed to be responsive to the first ALS therapy if the serum concentration of the immune-based biomarker decreases or remains within the reference concentration in at least one successive serum sample taken from the ALS patient.

In particular embodiments of the above method for treating ALS, in instances where a patient is found to not be responsive to the first ALS therapy, the method further comprises administering to the patient a second ALS therapy. In specific embodiments, the method may further comprise, following the administering the second ALS therapy to the ALS patient, assessing the responsiveness of the ALS patient to the second ALS therapy, and continuing to administer the second ALS therapy to the ALS patient if the ALS patient is assessed to be responsive to the second ALS therapy, wherein the responsiveness of the ALS patient to the ALS therapy comprises comparing the serum concentration of at least one immune-based biomarker to a reference concentration of the of least one immune-based biomarker, wherein the ALS patient is assessed to be responsive to the second ALS therapy if the serum concentration of the immune-based biomarker decreases or remains within the reference concentration in at least one successive serum sample taken from the ALS patient.

In certain embodiments, the first or second ALS therapy comprises administering one or more Treg infusions to the patient.

In certain embodiments, the first or second ALS therapy comprises administering a CTLA-4 fusion protein and IL-2 to the patient. In some embodiments, the CTLA-4 fusion protein is abatacept. In certain embodiments, the IL-2 is aldesleukin.

In certain embodiments, the first or second ALS therapy comprises administering one or more Treg extracellular vesicle (EV), e.g., exosome, infusions to the patient.

In certain embodiments, the responsiveness of the ALS patient to the ALS therapy comprises comparing the serum concentration of an immune-based biomarker to a reference concentration for a set of immune-based biomarkers, and the ALS patient is assessed to be responsive to the ALS therapy if the serum concentration of at least 50% of the immune-based biomarkers in the set decreases or remains within its reference concentration in at least one successive serum sample taken from the ALS patient. In some embodiments, the set of immune-based biomarkers comprises 1 immune-based biomarker. In certain embodiments, the set of immune-based biomarkers comprises 2 biomarkers. In certain embodiments, the set of immune-based biomarkers comprises 3 biomarkers. In certain embodiments, the set of immune-based biomarkers comprises 4 biomarkers. In certain embodiments, the set of immune-based biomarkers comprises 5 biomarkers. In certain embodiments, the set of immune-based biomarkers comprises 6 biomarkers. In certain embodiments, the set of immune-based biomarkers comprises 7 biomarkers. In certain embodiments, the set of immune-based biomarkers comprises 8 biomarkers. In certain embodiments, the set of immune-based biomarkers comprises 9 biomarkers. In certain embodiments, the set of immune-based biomarkers comprises 10 biomarkers.

In certain embodiments, the ALS patient is assessed to be responsive to the ALS therapy if the serum concentration of the immune-based biomarker decreases or remains within the reference concentration in at least 2 successive serum samples taken from the ALS patient. In certain embodiments, the ALS patient is assessed to be responsive to the ALS therapy if the serum concentration of the immune-based biomarker decreases or remains within the reference concentration in at least 3 successive serum samples taken from the ALS patient. In certain embodiments, the ALS patient is assessed to be responsive to the ALS therapy if the serum concentration of the immune-based biomarker decreases or remains within the reference concentration in at least 4 successive serum samples taken from the ALS patient. In certain embodiments, the ALS patient is assessed to be responsive to the ALS therapy if the serum concentration of the immune-based biomarker decreases or remains within the reference concentration in at least 5 successive serum samples taken from the ALS patient. In certain embodiments, the ALS patient is assessed to be responsive to the ALS therapy if the serum concentration of the immune-based biomarker decreases or remains within the reference concentration in at least 6 successive serum samples taken from the ALS patient. In certain embodiments, the ALS patient is assessed to be responsive to the ALS therapy if the serum concentration of the immune-based biomarker decreases or remains within the reference concentration in at least 7 successive serum samples taken from the ALS patient. In certain embodiments, the ALS patient is assessed to be responsive to the ALS therapy if the serum concentration of the immune-based biomarker decreases or remains within the reference concentration in at least 8 successive serum samples taken from the ALS patient. In certain embodiments, the ALS patient is assessed to be responsive to the ALS therapy if the serum concentration of the immune-based biomarker decreases or remains within the reference concentration in at least 9 successive serum samples taken from the ALS patient.

In certain embodiments, the ALS patient is assessed to be responsive to the ALS therapy if the serum concentration of the immune-based biomarker decreases or remains within the reference concentration in a majority of 3 or more successive serum samples taken from the ALS patient.

In certain embodiments, the immune-based biomarker comprises ox-LDL, OLR1, sCD14, LBP, CRP, 4-HNE, IL-17F, or IL-17C.

### 4. BRIEF DESCRIPTION OF THE DRAWINGS

FIG. 1A-1C: Biomarker concentrations as determined in individual serum samples of 8 untreated patients with rapidly progressing ALS (“Fast ALS patients”), 8 untreated patients with slowly progressing ALS (“Slow ALS patients”), and 9 age-matched healthy volunteers (“healthy controls”) for interleukin-17C (“IL-17C”) (FIG. 1A); interleukin-6 (“IL-6”) (FIG. 1B); and oxidized low-density lipoprotein receptor 1 (“OLR1”) (FIG. 1C). The horizontal bar represents the mean average value for each group.

FIG. 2A-2B: Results showing that oxidized low-density lipoprotein (“ox-LDL”) concentration in serum was elevated in sera from rapidly progressing patients (n=13) compared with slowly progressing patients or healthy controls; ox-LDL was not increased in sera from slowly progressing patients compared with healthy controls (FIG. 2A). Ox-LDL levels were increased in patients with Alzheimer's Disease (“AD”) or with mild cognitive impairment (“MCI”) (FIG. 2B). *<0.05, **<0.001, n.s.=not significant.

FIG. 3: Graph depicting ALS progression rate as assessed using points per month (“pts/mn”) on the amyotrophic lateral sclerosis functional rating scale (“ALSFRS”) in 30 untreated ALS patients versus serum immune-based biomarker ox-LDL concentration in serum.

FIG. 4A-4B: Graphs providing ALSFRS scores for 6 patients, subjects 205, 203, 202 (shown in FIG. 4A), and 206, 115, 114 (shown in FIG. 4B) over time from prior to administration of Treg infusions and after six Treg infusions (indicated by downward pointing arrows in upper panels), where the 6 patients were responsive to the ALS therapy. Dotted lines labeled “PRO-ACT” and “ceftriaxone” show ALSFRS scores from published sources of ALS patients in various other clinical trials. A decrease in ALSFRS score is indicative of disease progression.

FIG. 5: Graphs providing ALSFRS scores for 2 patients (subjects 201 and 103) over time from prior to administration Treg infusions and after six Treg infusions (indicated by downward pointing arrows), where the 2 patients were not responsive to the ALS therapy.

FIG. 6A-6C: Graphs showing IL-17F serum concentration (pg/mL) in non-responders (subjects 210 and 103 shown in FIG. 6A) and responders (subjects 202 and 114 shown in FIG. 6B and subjects 206 and 115 shown in FIG. 6C) in serum samples collected prior to administration of any Treg infusion (leftmost data point in each graph) and after administration of Treg infusions. Mean average IL-17F serum concentration for healthy controls is depicted in the graphs as a solid straight line in between parallel dashdotted lines showing one standard deviation above and below the mean average.

FIG. 7: Graph depicting the mean average IL-17F serum concentration for the non-responders and responders.

FIG. 8A-8B: Graphs showing IL-17C serum concentration (pg/mL) for responders (subjects 202, 203, 114 and 115 shown in FIG. 8A, and subject 205 and 206 shown in FIG. 8B). Serum samples were collected prior to administration of any Treg infusion (leftmost data point in each graph) and after administration of Treg infusions. Mean average IL-17C serum concentration for healthy controls is depicted in the graphs as a solid straight line in between dashdotted lines depicting one standard deviation above and below the mean average.

FIG. 9: Graphs showing IL-17C serum concentration (pg/mL) for non-responders. Serum samples were collected prior to administration of any Treg infusion (leftmost data point in each graph) and after administration of Treg infusions. Mean average IL-17C serum concentration for healthy controls is depicted in the graphs as a solid straight line in between dashdotted lines depicting one standard deviation above and below the mean average.

FIG. 10: Graph depicting the mean average IL-17C serum concentration (pg/mL) in non-responders an responders prior to administration of any Treg infusion (visit 1) and after administration of Treg infusions (visits 2-6), as compared to mean IL-17C serum concentration in healthy controls.

FIG. 11: Graph depicting mean average OLR1 serum concentration (pg/mL) in non-responders and responders after administration of Treg infusions as compared to mean OLR1 serum concentration in healthy controls.

FIG. 12: Graph depicting NF-L serum concentration (ng/mL) in a non-responder (subject 201) and two responders (subjects 202 and 203) prior to (visit 1) and after (visits 2-11) administration of Treg infusions as compared to mean OLR1 serum concentration in healthy controls.

FIG. 13: Graph depicting of mean average ox-LDL serum concentration (U/L) in non-responders and responders before (visit 1) and after (visits 2-6) administration of Treg infusions as compared to mean ox-LDL serum concentration in healthy controls.

FIG. 14: Graph depicting serum concentration of ox-LDL (upper panel), and ALSFRS score (lower panel) in an ALS patient over the course of weeks (x-axis) in which Treg infusions (indicated by the downward pointing arrows) were administered to the patient. Mean average ox-LDL serum concentration for healthy controls is depicted in the upper panel as a solid straight line in between dashdotted lines depicting one standard deviation above and below the mean average.

FIG. 15: Graph depicting serum concentration of ox-LDL (upper panel), and ALSFRS score (lower panel) in an ALS patient over the course of weeks (x-axis) in which Treg infusions (indicated by the downward pointing arrows) were administered to the patient. Mean average ox-LDL serum concentration for healthy controls is depicted in the upper panel as a solid straight line in between dashdotted lines depicting one standard deviation above and below the mean average.

FIG. 16: Graph depicting serum concentration of ox-LDL (upper panel), and AALS score (lower panel) in an ALS patient over the course of weeks (x-axis) in which Treg infusions (indicated by the downward pointing arrows) were administered to the patient. Mean average ox-LDL serum concentration for healthy controls is depicted in the upper panel as a solid straight line in between dashdotted lines depicting one standard deviation above and below the mean average.

FIG. 17A-17C: FIG. 17A: Graph depicting serum concentration of sCD14 (top panel), LBP (second from top panel), CRP (third from top panel), and ALSFRS score (bottom panel) in an ALS patient over the course of weeks (x-axis) in which Treg infusions (indicated by the downward pointing arrows) were administered to the patient. FIG. 17B: Graph depicting serum concentration of sCD14 (top panel), LBP (second from top panel), CRP (third from top panel), and ALSFRS score (bottom panel) in an ALS patient over the course of weeks (x-axis) in which Treg infusions (indicated by the downward pointing arrows) were administered to the patient. FIG. 17C: Graph depicting serum concentration of sCD14 (top panel), LBP (second from top panel), CRP (third from top panel), and ALSFRS score (bottom panel) in an ALS patient over the course of weeks (x-axis) in which Treg infusions (indicated by the downward pointing arrows) were administered to the patient. In each of FIGS. 17A to 17C: the mean average sCD14 serum concentration for healthy controls is depicted in the graphs as a solid straight line in between parallel lines depicting one standard deviation above and below the mean average.

FIG. 18A-18E: FIG. 18A: graph depicting ox-LDL serum concentrations in subject 1, who was one of 5 ALS patients administered with abatacept and IL-2 as a combination therapy. FIG. 18B: graph depicting ox-LDL serum concentrations in subject 2, who was one of 5 ALS patients administered with abatacept and IL-2 as a combination therapy. FIG. 18C: graph depicting ox-LDL serum concentrations in subject 3, who was one of 5 ALS patients administered with abatacept and IL-2 as a combination therapy. FIG. 18D: graph depicting ox-LDL serum concentrations in subject 4, who was one of 5 ALS patients administered with abatacept and IL-2 as a combination therapy. FIG. 18E: graph depicting ox-LDL serum concentrations in subject 5, who was one of 5 ALS patients administered with abatacept and IL-2 as a combination therapy. In each of FIG. 18A to 18E: the mean average ox-LDL serum concentration for healthy controls is depicted in the graphs as a solid straight line in between parallel lines depicting one standard deviation above and below the mean average.

FIG. 19A-19E: FIG. 19A: graph depicting 4-hydroxynonenal (“4-HNE”) serum concentrations in subject 1, who was one of 5 ALS patients administered with abatacept and IL-2 as a combination therapy. FIG. 19B: graph depicting 4-HNE serum concentrations in subject 2, who was one of 5 ALS patients administered with abatacept and IL-2 as a combination therapy. FIG. 19C: graph depicting 4-HNE serum concentrations in subject 3, who was one of 5 ALS patients administered with abatacept and IL-2 as a combination therapy. FIG. 19D: graph depicting 4-HNE serum concentrations in subject 4, who was one of 5 ALS patients administered with abatacept and IL-2 as a combination therapy. FIG. 19E: graph depicting 4-HNE serum concentrations in subject 5, who was one of 5 ALS patients administered with abatacept and IL-2 as a combination therapy. In each of FIGS. 19A to 19E: the mean average 4-HNE serum concentration for healthy controls is depicted in the graphs as a solid straight line in between parallel lines depicting one standard deviation above and below the mean average.

### 5. DETAILED DESCRIPTION

As demonstrated by the Examples presented herein, elevated concentrations of at least one serum immune-based biomarker in a biological sample from an ALS patient can be indicative of, e.g., likely progression of ALS in the ALS patient, and/or the ALS patient's likely responsiveness to an ALS therapy, and/or effectiveness of an ALS therapy being administered to the ALS patient.

As demonstrated by the results provided herein, serum levels of certain biomarkers are useful, for example, to monitor efficacy of, and responsiveness to, ALS therapy, e.g., ALS therapy that comprises infusing expanded regulatory T-lymphocytes (Tregs) into an ALS patient, and ALS therapy that comprises administering CTLA-4 fusion protein, e.g., abatacept, and IL-2, e.g., aldesleukin, to an ALS patient.

### Terminology

Unless specifically stated or apparent from context, as used herein, the terms “a”, “an”, and “the” are understood to be singular or plural, and denote “one or more.”

The terms “include,” “such as,” and the like are intended to convey inclusion without limitation, unless otherwise specifically indicated.

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. In particular, unless otherwise noted the terms mean within plus or minus 10% of a given value or range. In instances where an integer is required, the terms mean within plus or minus 10% of a given value or range, rounded either up or down to the nearest integer.

As used herein, the terms “treat”, “treating” and “treatment” may encompass therapeutic treatment, wherein the object is to prevent or slow down (lessen) an undesired physiological change associated with a disease or condition (e.g., ALS). Beneficial or desired clinical results include, but are not limited to, alleviation of symptoms, delay or slowing of the progression of a disease or condition (e.g., ALS), diminishment of the extent of a disease or condition (e.g., ALS), stabilization of a disease or condition (e.g., ALS), where the disease or condition (e.g., ALS) does not worsen, amelioration or palliation of the disease or condition (e.g., ALS), and remission (whether partial or total) of the disease or condition (e.g., ALS), whether detectable or undetectable. These term can also mean prolonging survival as compared to expected survival if not receiving treatment.

Unless denoted otherwise in the specific context in which they are used, the terms “individual,” “subject,” and “patient” are interchangeable as used herein and include but are not limited to a human. For example, in certain embodiments of the methods described herein, the individual, subject or patient is a mammal, such as, e.g., a non-human primate, dog, rabbit, rat, mouse, or goat. In certain embodiments, an individual, subject or patient is a human.

### ALS Patients

In embodiments of the methods provided herein, an “ALS patient” or a “patient diagnosed with ALS” is a patient is diagnosed with or is suspected of having Amyotrophic Lateral Sclerosis (ALS) or an ALS related disease or disorder.

As a non-limiting example, in some embodiments, the ALS or ALS related disorders can be selected from sporadic ALS, Familial ALS (FALS), Primary Lateral Sclerosis (PLS), limb-onset ALS, bulbar-onset ALS, primary lateral sclerosis, progressive muscular atrophy (PMA), Pseudobulbar Palsy and Progressive Bulbar Palsy (PBP), Fronto Temporal Dementia (FTD), and ALS-plus syndrome (e.g., in which patients exhibit additional symptoms beyond motor neuron involvement, including dementia, autonomic dysfunction, and sensory loss).

In some embodiments, a patient's ALS (e.g., disease status, disease progression, response to treatment) or ALS symptoms may be monitored using any clinical criteria disclosed herein or known in the art. Any assessment method or instrument (e.g., clinical rating scales) known in the art or disclosed herein may be used to monitor a patient's ALS disease or symptoms, such as, for instance, mini-mental state examination (MMSE), Norris scale, ALS severity scale, Appel ALS (AALS) rating, ALS Functional Rating Scale (ALSFRS), revised ALSFRS (ALSFRS-R), Accurate Test of Limb Isometric Strength (ATLIS); Combined Assessment of Survival and Function (CAFS), Electrical Impedance Myography (EIM), Hand-Held Dynamometry (HHD), Motor Unit Number Estimation (MUNE); Vital Capacity (VC), Forced Vital Capacity (FVC), Hasegawa dementia rating scale-revised (HDS-R), frontal assessment battery (FAB), Montreal cognitive assessment (MoCA), ALS-frontotemporal dementia-Questionnaire (ALS-FTD-Q), anosognosia scale, affective (depression, apathy, and behavioral and psychological symptoms of dementia (BPSD) assessments, and activities of daily living (ADL) assessments. Additional tools and methods for screening patients with ALS are known in the art and may be used, such as cognitive screeners, e.g., ACE-R, ALS-BCA, ALS-CBS, ECAS, FAB, MMSE, MoCA, PSSFTS, and UCSF-SB), and/or behavioral screeners, e.g., ALS-FTD-Q, AES, BBI, DAS, FBI, FrSBe, MIND-B, and NPI (see Gosselt et al., Amyotroph. Lateral. Scler. Frontotemporal. Degener., 21 (5-6): 324-336 (2020)).

The ALSFRS-R assesses bulbar (swallowing, speech), fine motor and gross motor functions, and breathing. The scale is from 0-48. In certain embodiments of the methods provided herein, progression of ALS is assessed by ALSFRS-R, where a decline of more than 0.5 points per month as assessed by ALSFRS-R, is a fast progression of ALS; if the ALS patient has decline of equal to or less than 0.5 points per month, the ALS is slow progressing ALS.

In certain embodiments, progression of ALS can be measured by FVC. In some embodiments, breathing declining more than 3% per month is fast progressing ALS; breathing declining equal to or less than 3% is slow progressing ALS.

In certain embodiments of the methods provided herein, ALS progression can be assessed by AALS, e.g., where fast progressing ALS patient decline at a rate of greater or equal to 1.5 AALS points/month, and slowly progressing patients progress at less than 1.5 AALS points/month.

Reference to an ALS patient as “responder” herein, can, for example, refer to an ALS patient responsive to an ALS therapy, e.g., Treg infusion(s), as demonstrated by an improvement or at least a non-decline over time in an instrument for assessing ALS progression, such as any of those mentioned herein (e.g., ALSFRS/ALSFRS-R, AALS, FVC, etc.). In certain embodiments, a “responder” is an ALS patient with fast progressing ALS (e.g., as explained above) prior to being administered an ALS therapy, e.g., Treg infusion(s), who has slowly progressing ALS after being administered the ALS therapy, e.g., Treg infusion(s). It will be understood that any such improvement or non-decline or slower rate with respect to ALS progression observed in a “responder” following the ALS therapy, e.g., Treg infusion(s), need not be permanent for the ALS patient to be a “responder.”

As used herein, a “non-responder” is a patient that does not exhibit responsiveness to an ALS therapy, e.g., Treg infusion(s), and will continue to decline in measures tracking progression of ALS (e.g., ALSFRS/ALSFRS-R, AALS, FVC, etc.) despite, e.g., additional Treg infusions as part of the Treg therapy.

It will be understood that while an ALS therapy, e.g., Treg infusion(s), when administered to a “responder” can have acceptable or good efficacy, the same ALS therapy, e.g., Treg infusion(s) when administered to a “non-responder” can have poor efficacy.

Without being bound by any theory or limitation, it is believed that serum levels of at least one serum immune-bases biomarker can remain elevated in some ALS patients that are given Treg therapy, and that such ALS patients will continue to decline in measures tracking progression of ALS despite, e.g., additional Treg infusions as part of a Treg therapy. For such patients, the ALS therapy is predicted to have poor efficacy.

### Biological Samples and Biomarkers

In embodiments of the methods provided herein, a concentration of one or more biomarkers is determined in a biological sample collected from a subject.

In certain embodiments the biological sample can be urine, cerebrospinal fluid, blood, or blood component (e.g., serum).

In certain embodiments, the biological sample is serum.

A biomarker can, for example, be a protein that has a detectable concentration that changes in a subject, typically in manner that correlates with an inflammatory state in the subject (or alterations in an inflammatory state), including, for example, neuroinflammation, peripheral immune alterations/inflammation, and so forth. A serum immune-based biomarker can, for example, be a protein with a concentration in the sera collected from a subject that is detectable at least during a time associated with an inflammation (e.g., neuroinflammation, peripheral immune alteration/inflammation, etc.).

In some embodiments of the methods provided herein, a concentration of at least one serum immune-based is determined relative to a reference concentration and/or is compared to a reference concentration. Exemplary biomarkers are disclosed herein, such as those described in the examples below. Any biomarkers disclosed herein may be used with the methods provided. In some embodiments, the biomarkers are detected and/or measured in the blood of a patient, or in a blood component (e.g., a serum sample).

In certain embodiments, the biomarker is oxidized low-density lipoprotein (ox-LDL). Measurement of ox-LDL levels in plasma or serum has been incorporated into clinical practice in the diagnosis and treatment of lipid disorders (such as diabetes mellitus), atherosclerosis, and various liver and renal diseases, especially as it pertains to the evaluation of oxidative stress. See, e.g., Nakhjavani et al., “Serum oxidized-LDL is associated with diabetes duration independent of maintaining optimized levels of LDL-cholesterol”, Lipids, 45 (4): 321-327 (2010); Steinberg, “Low density lipoprotein oxidation and its pathobiological significance”, J. Biol. Chem., 272 (34): 20963-20966 (1997). Serum ox-LDL testing services (e.g., Labcorp, Burlington NC) and kits (e.g., Oxidized LDL ELISA Kit (Fisher Scientific)) are commercially available.

In certain embodiments, the biomarker is oxidized low density lipoprotein receptor 1 (OLR1; see, e.g., UniProtKB/Swiss-Prot Accession P78380 for an exemplary OLR1 sequence). OLR1 is also known in the art as lectin-type oxidized LDL receptor 1, LOX-1, LOXIN, SLOX1, scavenger receptor class E member 1, SCARE1, among others. Serum OLR1 testing services (e.g., using the OLINK® platform (Olink, Boston MA)) and kits (e.g., Human LOX-1/OLR1 DuoSet ELISA (R&D Systems)) are commercially available.

In certain embodiments, the biomarker is soluble CD14 (sCD14). sCD14 is an acute phase protein, a class of proteins whose concentration in blood increase in response to inflammation. See, e.g., Bas et al., “CD14 is an Acute-Phase Protein”, J. Immunol., 172:4470-4479 (2004). Human sCD14 ELISA kits for measuring sCD14 levels are commercially available (e.g., Hycult Biotech Inc., Wayne PA; R&D Systems, Minneapolis MN)).

In certain embodiments, the biomarker is lipopolysaccharide binding protein (LBP; for an exemplary LBP sequence see, e.g., UniProtKB Accession P18428 LBP_Human). Human LBP ELISA kits for assaying LBP levels are commercially available (e.g., Catalog No. EH1560, Wuhan Fine Biotech Co., Ltd., Wuhan, China; and the LBP DuoSet ELISA Kit, R&D Systems).

In certain embodiments, the biomarker is neurofilament light chain (NF-L; for an exemplary NF-L sequence see, e.g., UniProtKB Accession P07196 NFL_HUMAN). NF-L immunoassay kits and assay systems are commercially available (e.g., the SIMPLE PLEX HUMAN NF-L CARTRIDGE for use in ELLA AUTOMATED IMMUNOASSAY SYSTEM, Bio-Techne, San Jose, CA), as are a number of services that can measure NF-L in serum samples (e.g., Labcorp, Burlington NC).

In certain embodiments, the biomarker is C Reactive Protein (CRP; for an exemplary CRP sequence see, e.g., UniProtKB Accession P02741 CRP_HUMAN). Numerous commercially kits and services are available for measuring CRP levels in serum samples (e.g., CRP Quantikine ELISA Kit, R&D Systems).

In certain embodiments, the biomarker is 4-hydroxynonenal (4-HNE; CAS Reg. No. 75899-68-2). A number of immunoassays to measure 4-HNE levels in sera are commercially available. In addition, since 4-HNE displays a relatively fast half-life of less than 2 minutes in normal physiological conditions, assays can detect levels of protein adducts of 4-HNE as a surrogate for 4-HNE. ELISA kits are commercially available for protein adducts of 4-HNE. An assay for determining 4-HNE adduct levels in serum is described, for instance, in Monroe et al., “A Highly Sensitive, Reproducible Assay for Determining 4-hydroxynonenal Protein Adducts in Biological Material”, Bio Protoc. 9 (19): e3383 (2019).

In certain embodiments, the biomarker is interleukin 6 (IL-6; for an exemplary IL-6 sequence see, e.g., UniProtKB Accession P05231 IL6_HUMAN). Quantitative detection of IL-6 in serum can be conducted using commercially available reagents, kits and platforms (e.g., ELECSYS IL-6 immunoassay for use on COBAS E immunoassay analyzers, Roche Diagnostics, Indianapolis IN).

In certain embodiments, the biomarker is interleukin 17F (IL-17F; for an exemplary IL-17F sequence see, e.g., UniProtKB Accession Q96PD4 IL17F_HUMAN). IL-17F levels in serum can be determined using, for instance, the ALPHALISA Human IL-17F Detection Kit (Perkin Elmer, Santa Clara CA).

In certain embodiments, the biomarker is interleukin 17C (IL-17C; for an exemplary IL-17C sequence see, e.g., UniProtKB Accession Q9P0M4 IL17C_HUMAN). An exemplary commercially available kit for detecting IL-17C is serum is the SIMOA IL-17C Discovery Kit (Quanterix, Billerica MA).

In certain embodiments, the serum sample is collected from the patient prior to any ALS therapy.

In certain embodiments, the serum sample is collected from the patient prior to any Treg infusion.

In certain embodiments, the serum sample is collected from the patient after a Treg infusion has been administered to the patient.

In certain embodiments, the serum sample is collected from the patient on the day following the Treg infusion.

In some embodiments of the methods provided herein, the at least one serum immune-based biomarker comprises ox-LDL, OLR1, sCD14, LBP, NF-L, CRP, 4-HNE, IL-6, IL-17F, or IL-17C.

In some embodiments of the methods provided herein, the at least one serum immune-based biomarker comprises ox-LDL, OLR1, sCD14, LBP, CRP, IL-17F, or IL-17C.

In some embodiments of the methods provided herein, the at least one serum immune-based biomarker comprises ox-LDL, OLR1, sCD14, LBP, IL-17F, or IL-17C.

In some embodiments of the methods provided herein, the at least one serum immune-based biomarker comprises ox-LDL, OLR1, NF-L, IL-17F, or IL-17C.

In some embodiments of the methods provided herein, the at least one serum immune-based biomarker comprises ox-LDL, OLR1, IL-17F, or IL-17C.

In some embodiments of the methods provided herein, the at least one serum immune-based biomarker comprises ox-LDL, OLR1, sCD14, or LBP.

In certain embodiments, the at least one serum immune-based biomarker comprises ox-LDL. In certain embodiments, the at least one serum immune-based biomarker comprises OLR1. In certain embodiments, the at least one serum immune-based biomarker comprises sCD14. In certain embodiments, the at least one serum immune-based biomarker comprises IL-17C. In certain embodiments, the at least one serum immune-based biomarker comprises IL-17F. In certain embodiments, the at least one serum immune-based biomarker comprises LBP.

In certain embodiments, the at least one serum immune-based biomarker comprises 4-HNE.

In certain embodiments, the at least one serum immune-based biomarker is selected from the group consisting of ox-LDL, OLR1, sCD14, LBP, NF-L, IL-6, IL-17F, and IL-17C. In certain embodiments, the at least one serum immune-based biomarker is selected from the group consisting of ox-LDL, OLR1, sCD14, LBP, IL-17F, and IL-17C. In certain embodiments, the at least one serum immune-based biomarker is selected from the group consisting of ox-LDL, OLR1, sCD14, LBP, IL-6, IL-17F, and IL-17C.

In certain embodiments, the at least one serum immune-based biomarker is selected from the group consisting of ox-LDL, OLR1, NF-L, IL-17F, and IL-17C. In certain embodiments, the at least one serum immune-based biomarker is selected from the group consisting of ox-LDL, OLR1, IL-17F, and IL-17C. In certain embodiments, the at least one serum immune-based biomarker is selected from the group consisting of ox-LDL, OLR1, sCD14, and LBP. In certain embodiments, the at least one serum immune-based biomarker is selected from the group consisting of ox-LDL, OLR1, sCD14, IL-17F, and IL-17C.

In certain embodiments, the at least one serum immune-based biomarker is selected from the group consisting of ox-LDL, OLR1, sCD14, LBP, NF-L, IL-6, 4-HNE, IL-17F, and IL-17C.

In certain embodiments, the at least one serum immune-based biomarker is selected from the group consisting of ox-LDL, and OLR1.

In certain embodiments, the at least one serum immune-based biomarker is selected from the group consisting of ox-LDL, and 4-HNE.

In some embodiments of the methods provided, concentration of a single serum immune-based biomarker is determined and/or compared to a reference concentration. In certain embodiments, concentrations of at least two serum immune-based biomarkers are each determined and/or compared to a reference concentration. In certain embodiments, concentrations of two serum immune-based biomarkers are each determined and/or compared to a reference concentration. In certain embodiments, concentrations of at least three serum immune-based biomarkers are each determined and/or compared to a reference concentration. In certain embodiments, concentrations of at least four serum immune-based biomarkers are each determined and/or compared to a reference concentration. In certain embodiments, concentrations of at least five serum immune-based biomarkers are each determined and/or compared to a reference concentration.

### Reference Concentration

A concentration of a biomarker can be determined to be elevated if the concentration of the biomarker in a sample, when compared to a reference concentration, is greater than a reference concentration. In exemplary methods described herein, a concentration of at least one serum immune-based biomarker is determined and/or compared to a reference concentration.

It will be understood that a given biomarker concentration is compared to a reference concentration specific to that biomarker. For example, an ox-LDL concentration is compared to a reference concentration for ox-LDL; an IL-17F concentration is compared to a reference concentration for IL-17F, and so forth for any given biomarker.

In certain embodiments, a reference concentration is obtained from healthy individuals.

In certain embodiments, a reference concentration is a mean average concentration from healthy individuals.

In some embodiments, a reference concentration is one standard deviation above the mean average concentration from a group of healthy individuals.

In some embodiments, a reference concentration is a range of concentrations encompassing an mean average concentration from a group of healthy individuals.

The number of healthy individuals that contribute to a reference concentration can, for example, be at least 3. In certain embodiments, the number of healthy individuals is between 3 to about 10,000. In other embodiments, the number of healthy individuals is between 5 to about 1,000, or between 8 to 40. In some embodiments, the number healthy individuals contributing to the reference concentration is at least 4, at least 5, at least 6, at least 7, at least 8, at least 9, at least 10, at least 15, at least 20, at least 25, at least 30, at least 35, at least 40, at least 50, at least 75, at least 100, at least 150, at least 200, at least 250, at least 300, at least 400, at least 500, at least 750, at least 1,000, at least 2,000, or at least 3,000.

In certain embodiments, the reference concentration is from healthy individuals that are age-matched to the ALS patient.

In some embodiments, a reference concentration is a range of concentrations encompassing a mean average concentration from a group of ALS patients responsive to Treg therapy. In some embodiments, a reference concentration is a range of concentrations encompassing a mean average concentration from a group of ALS patients responsive to Treg therapy plus one standard deviation above and below the mean average concentration.

In certain embodiments, where a reference concentration is a range, a concentration of a serum immune-based biomarker that is within that range, including the upper and lower limits of the range, is the same as, or at, the reference concentration.

In certain embodiments of the methods provided, the reference concentration for the at least one serum immune-based biomarker comprising ox-LDL, OLR1, sCD14, LBP, NF-L, CRP, 4-HNE, IL-6, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| NF-L | 0.88 ± 0.21 | ng/ml |
| CRP | 1.08 ± 1.04 | ug/mL |
| 4-HNE | 5 | ug/mL |
| IL-6 | 2.45 ± 1.49 | pg/mL |
| IL-17F | 0.34 ± 0. | 54 pg/mL |
| IL-17C | 8.47 ± 3.08 | pg/mL. |

In certain embodiments, a reference concentration range can be as follows:

| | |
|---|---|
| ox-LDL | from about 48 U/L to about 100 U/L; from about 50 U/L to |
| | about 70 U/L; or from about 70 U/L to about 90 U/L. |
| OLR1 | from about 150 pg/mL to about 450 pg/mL; from about 160 |
| | pg/mL to about 425 pg/mL; or from about 250 pg/mL to |
| | about 400 pg/mL. |
| sCD14 | from about 1.9 ug/mL to about 3.1 ug/mL; from about 2 |
| | ug/mL to about 2.9 ug/mL; or from about 2.1 ug/mL to |
| | about 2.7 ug/mL. |
| LBP | from about 5 ug/mL to about 30 ug/mL; or from about 10 |
| | ug/mL to about 25 ug/mL. |
| NF-L | from about 0.3 ng/ml to about 1.1 ng/ml; from about 0.3 |
| | ng/ml to about 0.6 ng/ml; or from about 0.4 ng/ml to about |
| | 0.8 ng/ml. |
| CRP | from about 0.3 ug/mL to about 2.2 ug/mL; from about 0.5 |
| | ug/mL to about 2.0 ug/mL; or from about 1 ug/mL to about |
| | 2.0 ug/mL. |
| 4-HNE | From about 0.5 ug/mL to about 12 ug/mL; from about 1 |
| | ug/mL to about 10 ug/mL; or from about 2 ug/mL to about |
| | 9 ug/mL. |
| IL-6 | from about 0.8 pg/mL to about 4.1 pg/mL; from about 2.8 |
| | pg/mL to about 4 pg/mL; or from about 3.2 pg/mL to about |
| | 3.7 pg/mL. |
| IL-17F | from below a level of detection to about 4.5 pg/mL; from |
| | below a level of detection to about 1 pg/mL; from about 0.01 |
| | pg/mL to about 2.0 pg/mL; from about 0.01 pg/mL to about |
| | 1.0 pg/mL. |
| IL-17C | from about 4 pg/mL to about 19 pg/mL; from about 5 pg/mL |
| | to about 14 pg/mL; from about 5 pg/mL to about 11 pg/mL; |
| | or from about 7 pg/mL to about 15 pg/mL. |

In certain embodiments, a reference concentration for ox-LDL, can be 48 U/L, 50 U/L, 52 U/L, 56.8 U/L, 60 U/L, 61 U/L, 63.8 U/L, 70 U/L, 75 U/L, 80 U/L, 90 U/L, or 100 U/L.

In certain embodiments, a reference concentration for OLR1 can be 150 ng/ml, 160 ng/mL, 200 pg/mL, 250 pg/mL, 280 pg/mL, 290 pg/mL, 297 pg/mL, 299.6 pg/mL, 427.7 pg/mL, 430 pg/mL, 350 pg/mL, 360 pg/mL, 375 pg/mL, 400 pg/mL, 415 pg/mL, 425 pg/mL, 425 pg/mL, or 450 pg/mL.

In certain embodiments, a reference concentration for sCD14 can be 1.9 ug/mL, 2 pg/mL, 2.1 ug/mL, 2.25 ug/mL, 2.56 ug/mL, 2.7 ug/mL, 2.9 ug/mL, or 3 pg/mL.

In certain embodiments, a reference concentration for LBP can be 5 pg/mL, 7 pg/mL, 9 pg/mL, 10 pg/mL, 12 pg/mL, 15 pg/mL, 17 pg/mL, 20 pg/mL, 20.18 ug/mL, 25 pg/mL, 27.66 ug/mL, or 30 pg/mL.

In embodiments of the method provided herein, a reference concentration for NF-L can be 0.3 ng/ml, 0.4 ng/ml, 0.41 ng/ml, 0.5 ng/mL, 0.6 ng/mL, 0.62 ng/ml, 0.7 ng/mL, 0.75 ng/mL, 0.79 ng/mL, 0.8 ng/mL, 0.82 ng/mL, 0.85 ng/mL, 0.88 ng/mL, or 1.09 ng/mL.

In certain embodiments, a reference concentration for CRP can be 0.3 ug/mL, 0.5 ug/mL, 1 pg/mL, 1.08 ug/mL, 1.5 ug/mL, 1.8 ug/mL, 2 pg/mL, or 2.12 ug/mL.

In certain embodiments, a reference concentration for 4-HNE can be 0.5 ug/mL, 1 pg/mL, 1.5 ug/mL, 2.0 ug/mL, 2.5 ug/mL, 3.0 ug/mL, 3.5 ug/mL, 4 pg/mL, 4.5 ug/mL, 5 pg/mL, 5.5 ug/mL, 6 pg/mL, 6.5 ug/mL, 7 pg/mL, 7.5 ug/mL, 8 pg/mL, 8.5 ug/mL, 9 pg/mL, 9.5 ug/mL, 10 pg/mL, 10.5 ug/mL, 11 pg/mL, 11.5 ug/mL, or 12 pg/mL.

In certain embodiments, a reference concentration for IL-6 can be 2.46 pg/mLm 2.5 pg/mL, 2.8 pg/mL, 3 pg/mL, 3.2 pg/mL, 3.5 pg/mL, 3.7 pg/mL, 3.94 pg/mL, 4 pg/mL, or 4.1 pg/mL.

In certain embodiments, a reference concentration for IL-17F can be 0.01 pg/mL, 0.2 pg/mL, 0.27 pg/mL, 0.34 pg/mL, 0.5 pg/mL, 0.88 pg/mL, 1 pg/mL, 1.1 pg/mL, 1.5 pg/mL, 2 pg/mL, 2.45 pg/mL, 2.5 pg/mL, 3 pg/mL, 4 pg/mL, or 4.5 pg/mL.

In certain embodiments, a reference concentration for IL-17C can be 5 pg/mL, 5.5 pg/mL, 6 pg/mL, 7 pg/mL, 7.8 pg/mL, 8 pg/mL, 8.47 pg/mL, 10 pg/mL, 10.2 pg/mL, 11 pg/mL, 11.55 pg/mL, 12 pg/mL, 14 pg/mL, 15 pg/mL, or 19 pg/mL.

It will be understood that a reference concentration used for any given biomarker is independent of the reference concentration used for any other biomarker.

In certain embodiments of the methods provided herein, the reference concentration for each of the serum immune-based biomarkers can be as follows:

| | Reference | |
|---|---|---|
| Biomarker | Concentration | |
| ox-LDL | 90 | U/L |
| OLR1 | 450 | pg/mL |
| sCD14 | 2.7 | ug/mL |
| LBP | 30 | ug/mL |
| NF-L | 0.8 | ng/ml |
| CRP | 2.0 | ug/mL |
| 4-HNE | 5 | ug/mL |
| IL-6 | 4.0 | pg/mL |
| IL-17F | 2.0 | pg/mL |
| IL-17C | 14.0 | pg/mL. |

In certain embodiments, the reference concentration for each of the serum immune-based biomarkers is selected from any of the following sets of reference concentrations:

| | Exemplary | | Exemplary | | Exemplary | | Exemplary | |
|---|---|---|---|---|---|---|---|---|
| | Reference | | Reference | | Reference | | Reference | |
| | Concen- | | Concen- | | Concen- | | Concen- | |
| Bio- | tration | | tration | | tration | | tration | |
| marker | (Set 1) | | (Set 2) | | (Set 3) | | (Set 4) | |
| IL- | 0.01 | pg/mL | 0.27 | pg/mL | 1.1 | pg/mL | 2.0 | pg/mL |
| 17F | | | | | | | | |
| OLR1 | 160 | pg/mL | 297 | pg/mL | 415 | pg/mL | 450 | pg/mL |
| NF-L | 0.41 | ng/mL | 0.62 | ng/mL | 0.79 | ng/mL | 0.8 | ng/mL |
| ox- | 52 | U/L | 61 | U/L | 70 | U/L | 90 | U/L |
| LDL | | | | | | | | |
| IL- | 5.5 | pg/mL | 7.8 | pg/mL | 10.2 | pg/mL | 14 | pg/mL |
| 17C | | | | | | | | |
| sCD14 | 2.0 | ug/mL | 2.25 | ug/mL | 2.5 | ug/mL | 2.7 | ug/mL |
| LBP | 15 | ug/mL | 20 | ug/mL | 25 | ug/mL | 30 | ug/mL |
| CRP | 0.5 | ug/mL | 1.0 | ug/mL | 1.8 | ug/mL | 2.0 | ug/mL |
| IL-6 | 2.8 | pg/mL | 3.2 | pg/mL | 3.7 | pg/mL | 4.0 | pg/mL |

In some embodiments of the method provided herein, a concentration of at least one biomarker is compared to a reference concentration, wherein the reference concentration is a concentration in a biological sample from the ALS patient prior to being administered an ALS therapy. In certain embodiments, the reference concentration is a concentration in a serum sample collected from the ALS patient prior to being administered an ALS therapy. In certain embodiments, the reference concentration is a concentration in a serum sample collected from the ALS patient prior to being administered any Treg infusion.

In yet other embodiments, the reference concentration is a concentration in serum sample collected from the ALS patient after a Treg infusion.

Any technique known in the art may be employed to determine biomarker concentration in a biological sample. For instance, concentration of a biomarker can, for example, be determined by immunoassay. Concentration of a biomarker in a biological sample can, for example, be determined using an enzyme-linked immunosorbent assay (ELISA). The ELISA is a well-known and commonly used analytical biochemical assay. Concentration of a biomarker can, for example, be determined using the PROXIMITY EXTENSION ASSAY (PEA) technology used in OLINK panels (Olink Proteomics, Watertown, MA).

In certain embodiments, measurements of ox-LDL concentrations expressed in units per liter (U/L) are made using the monoclonal antibody 4E6, for example, as described in Holvoet et al., Clinical Chemistry, 52 (4): 760-764 (2006), which is incorporated herein by reference in its entirety for all purposes. A kit based on the 4E6 monoclonal antibody is commercially available from Mercodia (Uppsala, Sweden). In certain embodiments, one arbitrary unit of ox-LDL immunoreactivity is equivalent to 300 ng.

In some embodiments of the methods provided herein, the concentration of the one or more serum immune-based biomarkers is determined using an enzyme-linked immunosorbent assay (ELISA).

### ALS Therapy

In certain embodiments of the methods provided herein such as, for instance, methods of selecting an ALS patient for an ALS therapy, methods for predicting an ALS patient's likely responsive to an ALS therapy, methods for monitoring efficacy of an ALS therapy, and the like, the ALS therapy can be any therapy that is administered to an ALS patient to treat ALS.

In certain embodiments of methods provided herein, the method can comprise administering the ALS therapy to the ALS patient to treat ALS.

In some embodiments of methods provided herein, the method can comprise administering the ALS therapy to the ALS patient in a clinical trial to test the ALS therapy.

In certain embodiments, an ALS therapy can, for example, be riluzole (RILUTEK®), TIGLUTIK (thickened riluzole), EXSERVAN™ (riluzole oral film), BHV-0223 (sublingual riluzole), NUEDEXTA® (dextromethorphan HBr and quinidine sulfate), ravulizumab-cwvz (ULTOMIRIS®), mesenchymal stem cell (MSC)-neurotrophic factor (NTF) cells (e.g., NUROWN®), MASITINIB (an oral tyrosine kinase inhibitor), TOFERSEN (BIIB067, IONIS-SODIRx, Ionis Pharmaceuticals and Biogen), RADICAVA™ (edaravone), AMX0035 (Amylyx), APB-102 (Apic Bio), H.P. ACTHAR GEL (Mallinckrodt Pharmaceuticals), MN-166 (MediciNova), GM-6 (Genervon), GILENYA (fingolimod, ALS TDI), ARIMOCLOMOL (Orph-001,Orphazyme), NP001 (Neuraltus), VM202 (VM Biopharma), RELDESEMTIV (Cytokinetics), NUROWN (BrainStorm Cell Therapeutics), NSI-566 (Neuralstem), MEXILETINE, acamprosate, baclofen, cinacalcet, sulfisoxazole, or torasemide.

In certain embodiments of the methods provided herein, the ALS therapy is interleukin-2 (“IL-2”) which can be administered alone or in combination with a second agent. In certain embodiments, the IL-2 is aldesleukin.

In some embodiments the ALS therapy is IL-2 and CTLA-4 fusion protein, e.g., abatacept.

Exemplary methods of administering IL-2 and abatacept combination therapy are described, for example, in International Application No. PCT/US2022/019748, which is incorporated herein by reference for its teaching of such methods.

In certain embodiments of the methods provided herein, the ALS therapy is a Treg therapy.

In certain embodiments, the ALS therapy comprises a Treg infusion.

In certain embodiments, the ALS therapy comprises a plurality of Treg infusions.

Administering Treg therapy to ALS patients has been shown to slow progression rates of the disease, and Treg suppressive function in some ALS patients correlates with the slowing of disease progression (Thonhoff, J. R. et al., 2018, Neurology-Neuroimmunology Neuroinflammation 5 (4): e465 (2018)). However, as demonstrated in the Examples below, not all ALS patients are responsive to Treg therapy. In one aspect, methods are provided herein to stratify patients that will likely be, or are, responsive to Treg therapy (“responders”), and those that will likely not be, or are not, responsive to Treg therapy (“non-responders”).

Certain methods provided herein may be performed with any steps of isolating, expanding, and administering Treg therapy to subjects (e.g., ALS patients) that are known in the art or disclosed herein. Exemplary methods of producing obtaining, enriching for and ex-vivo expanding a population of Tregs are described in International PCT Publication No. WO2021113685A2, which is incorporated by reference herein in its entirety, in particular for its teaching of such methods.

As a non-limiting example, in some embodiments, the ALS therapy comprises collecting white blood cells from the ALS patient (leukapheresis); isolating and expanding ex vivo Tregs from the collected white blood cells; and administering the expanded Tregs intravenously (infusion) to the ALS patient.

In certain embodiments of the methods provided herein, a single Treg infusion is administered to the ALS patient. In some embodiments of the methods provided herein, a plurality of Treg infusions are administered to the ALS patient.

In particular embodiments, the Tregs may be administered with IL-2. In certain embodiments, IL-2 is administered followed by Treg administration. In some embodiments, Treg administration (e.g., infusion) can be administered concomitantly with IL-2, e.g., with subcutaneous IL-2 injection(s).

In some embodiments the ALS therapy comprises anti-inflammatory and restorative extracellular vesicles (EVs) derived from ex vivo-expanded Tregs. Exemplary methods for preparing EVs, and for administering EV therapy to patients, are described, for example, in International Application No. PCT/US2022/017990, which is incorporated herein in its entirety, in particular for its teaching of such compositions and methods.

### Methods

In one aspect, provided herein are methods for selecting a patient for amyotrophic lateral sclerosis (ALS) therapy.

In some embodiments, a method for selecting a patient for ALS therapy comprises: (a) determining a concentration of interleukin 17F (IL-17F) in a serum sample collected from a patient diagnosed with or suspected of having ALS, wherein if the IL-17F concentration in the serum sample is at least 2.0 pg/mL the patient is excluded from a treatment with the ALS therapy, otherwise the patient is selected for the treatment with the ALS therapy; and (b) administering the ALS therapy to the selected patient.

In one aspect, provided herein are methods of treating ALS in a patient.

In some embodiments, a method of treating ALS comprises: administering an ALS therapy to a patient diagnosed with ALS, wherein it has been determined that a serum sample collected from the patient contains an IL-17F concentration of less than 2.0 pg/mL.

In some embodiments, a method for selecting a patient for ALS therapy comprises: (a) determining whether a concentration of at least one serum immune-based biomarker in a serum sample collected from a patient diagnosed with or suspected of having ALS is less than, equal to, or greater than, a reference concentration, wherein the at least one serum immune-based biomarker is IL-17F, oxidized low-density lipoprotein receptor 1 (OLR1), neurofilament light chain (NF-L), oxidized low-density lipoprotein (ox-LDL), or interleukin 17C (IL-17C); and wherein if the concentration of the at least one serum immune-based biomarker is greater than the reference concentration, the patient is excluded from a treatment with the ALS therapy, otherwise the patient is selected for the treatment with the ALS therapy; and (b) administering the ALS therapy to the selected patient.

In some embodiments, a method of treating ALS comprises: administering an ALS therapy to a patient diagnosed with ALS, wherein it has been determined that a serum sample collected from the patient contains a concentration of at least one serum immune-based biomarker that is less than, or equal to, a reference concentration, wherein the at least one serum immune-based biomarker is IL-17F, OLR1, NF-L, ox-LDL, or IL-17C.

In some embodiments of the methods provided herein, a reference concentration for the at least one biomarker comprising ox-LDL, OLR1, NF-L, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| NF-L | 0.88 ± 0.21 | ng/ml |
| IL-17F | 0.34 ± 0.54 | pg/mL |
| IL-17C | 8.47 ± 3.08 | pg/mL |

In one aspect, provided herein are methods of monitoring efficacy of Treg therapy to treat ALS.

In some embodiments, a method of monitoring efficacy of a Treg therapy comprises: (a) administering a Treg therapy to a patient diagnosed with ALS, wherein the Treg therapy comprises one or more Treg infusions; and (b) determining whether a concentration of at least one serum immune-based biomarker in a serum sample collected from the patient after a Treg infusion is less than, equal to, or greater than, a reference concentration, wherein the at least one serum immune-based biomarker comprises: ox-LDL, OLR1, sCD14, LBP, CRP, 4-HNE, IL-17F, or IL-17C; wherein the ALS therapy has poor efficacy where the concentration of the at least one serum immune-based biomarker is greater than its reference concentration.

In some embodiments, the ALS therapy has poor efficacy where the concentration of the at least one serum immune-based biomarker is more than 2-fold greater, more than 3-fold greater, more than 4-fold greater, more than 5-fold greater, more than 6-fold greater, more than 7-fold greater, more than 8-fold greater, more than 9-fold greater, more than 10-fold greater, or more than 20-fold greater than its reference concentration.

In some embodiments, a method of treating ALS comprises: (a) administering a Treg infusion to a patient diagnosed with ALS; (b) comparing a concentration of at least one serum immune-based biomarker in a serum sample obtained from the patient after the Treg infusion to a reference concentration, wherein the at least one immune-based biomarker comprises ox-LDL, OLR1, sCD14, LBP, CRP, 4-HNE, IL-17F, or IL-17C; and (c) administering an ALS therapy comprising a plurality of Treg infusions to the patient if the concentration of the at least one serum immune-based biomarker is equal to or below the reference concentration.

In some embodiments, provided herein is a method for treating a patient with a Treg therapy wherein the patient is suffering from ALS.

In certain embodiments, the method comprises: (a) administering to the patient a Treg therapy comprising Treg infusions being administered to the patient on different days; (b) comparing concentrations of at least one serum immune-based biomarker in serum samples to a reference concentration, wherein each of the serum sample is obtained from the patient after a Treg infusion, wherein the at least one serum immune-based biomarker comprises ox-LDL, OLR1, sCD14, LBP, CRP, 4-HNE, IL-17F, or IL-17C; and (c) maintaining the patient on the Treg therapy comprising administering Treg infusions following step (b), if the concentrations of the at least one serum immune-based biomarker is at, or less than, the reference concentration in all or at least 50% of the serum samples.

In some embodiments, provided herein is a method for treating ALS in a patient diagnosed therewith, the method comprising: (a) determining whether the concentration of at least one serum immune-based biomarker in a serum sample obtained from a patient diagnosed with ALS is at or below a reference concentration of the at least one serum immune-based biomarker, wherein the serum sample is obtained from the patient after being administered with a Treg infusion, wherein the at least one serum immune-based biomarker comprises ox-LDL, OLR1, sCD14, LBP, CRP, 4-HNE, IL-17F, or IL-17C; and (b) administering to the patient a Treg therapy comprising a plurality of Treg infusions if the concentration of the at least one serum immune-based biomarker is determined to be at or below its reference concentration.

In some embodiments of the methods provided, the reference concentration for the at least one serum immune-based biomarker is obtained from healthy individuals.

In other embodiments of the methods provided, the reference concentration for the at least one serum immune-based biomarker is a concentration in a serum sample obtained from the patient prior to any Treg infusion being administered to the patient.

In yet other embodiments of the methods provided, the reference concentration for the at least one serum immune-based biomarker comprising ox-LDL, OLR1, sCD14, LBP, CRP, 4-HNE, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| SCD14 | 2.56 ± 0.44 | ug/mL |
| LBP | 20.18 ± 7.48 | ug/mL |
| CRP | 1.08 ± 1.04 | ug/mL |
| 4-HNE | 5 | ug/mL |
| IL-17F | 0.34 ± 0.54 | pg/mL |
| IL-17C | 8.47 ± 3.08 | pg/mL. |

In one aspect, provided herein are methods for predicting a patient's likely responsiveness to an ALS therapy.

In some embodiments, the method comprises (a) collecting a serum sample from a patient diagnosed with ALS; and (b) comparing a concentration of at least one serum immune-based biomarker to a reference concentration; wherein the patient is predicted as likely to be non-responsive to the ALS therapy where the concentration of the at least one serum immune-based biomarker which is greater than its reference concentration.

In certain embodiments, the serum sample is collected from the patient after a Treg infusion.

In certain embodiments, the method further comprises enrolling the patient in a clinical trial to test an ALS therapy if the concentration of the at least one serum immune-based biomarker is the same or less than its reference concentration, or is in or below its reference concentration range.

In certain embodiments, the method further comprises administering the ALS therapy to the patient if the concentration of the at least one serum immune-based biomarker is the same or less than its reference concentration, or is in or below its reference concentration range.

In certain embodiments, if the concentration of the at least one biomarker is greater than its reference concentration, the patient is predicted to be non-responsive to ALS therapy. In some embodiments, if the concentration of the at least one biomarker is more than 2-fold greater, more than 3-fold greater, more than 4-fold greater, more than 5-fold greater, more than 6-fold greater, more than 7-fold greater, more than 8-fold greater, more than 9-fold greater, more than 10-fold greater, or more than 20-fold greater than its reference concentration, the patient is predicted as likely to be non-responsive to ALS therapy.

In one aspect, provided herein is a method of assessing the likely progression of ALS in a patient diagnosed with ALS.

In some embodiments, the method comprises comparing a concentration of at least one serum immune-based biomarker in a serum sample obtained from the patient diagnosed with ALS to a reference concentration obtained from healthy individuals; and assessing the likely progression of ALS to be fast if the concentration of the at least one serum immune-based biomarker is elevated relative to a reference concentration, and assessing the likely progression of ALS to be slow if the concentration of the at least one serum immune-based biomarker is at or below its referenced concentration.

In certain embodiments, the method further comprises administering an ALS therapy to the patient if the ALS is assessed to have fast progression.

In certain embodiments provided herein are methods for treating an ALS patient comprising administering an ALS therapy to the ALS patient; assessing the responsiveness of the ALS patient to the ALS therapy; and (i) continue administering the ALS therapy to the ALS patient if the ALS patient is assessed to be responsive to the ALS therapy, or (ii) discontinue administering the ALS therapy to the ALS patient if the ALS patient is assessed to be non-responsive to the ALS therapy; wherein the responsiveness of the ALS patient to the ALS therapy comprises comparing the serum concentration of an immune-based biomarker to a reference concentration and the ALS patient is assessed to be responsive to the ALS therapy if the serum concentration of the immune-based biomarker decreases or remains within the reference concentration in successive serum samples taken from the ALS patient, otherwise the ALS patient is assessed to be non-responsive to the ALS therapy.

In certain embodiments, the responsiveness of the ALS patient to the ALS therapy comprises comparing the serum concentration of an immune-based biomarker to a reference concentration for each immune-based biomarker in a set of two or more immune-based biomarkers, and the ALS patient is assessed to be responsive to the ALS therapy if the serum concentration of each immune-based biomarker in the set of two or more immune-based biomarkers decreases or remains within the reference concentration in successive serum samples taken from the ALS patient, otherwise the ALS patient is assessed to be non-responsive to the ALS therapy. In some embodiments, the set of two or more immune-based biomarkers comprises 2, 3, 4, 5, 6, 7, 8, 9, or 10 immune-based biomarkers.

In certain embodiments, the responsiveness of the ALS patient to the ALS therapy comprises comparing the serum concentration of an immune-based biomarker to a reference concentration for each immune-based biomarker in a set of immune-based biomarkers, and the ALS patient is assessed to be responsive to the ALS therapy if the serum concentration of at least 50% of the immune-based biomarkers in the set of immune-based biomarkers decreases or remains within the reference concentration in successive serum samples taken from the ALS patient, otherwise the ALS patient is assessed to be non-responsive to the ALS therapy. In some embodiments, the set of immune-based biomarkers comprises 1, 2, 3, 4, 5, 6, 7, 8, 9, or 10 immune-based biomarkers.

It will be understood that “successive serum samples” means serum samples taken from the ALS patient on different days, e.g., on different visits to a hospital, clinician's office or wherever blood samples are taken from the ALS patient. For example, “at least one successive serum sample” refers to a serum sample taken on a day following the day on which in initial or first serum sample was taken from the ALS patient. In certain embodiments, successive serum samples comprise at least two serum samples. In certain embodiments, successive serum samples comprise at least three serum samples. In certain embodiments, successive serum samples comprise at least four serum samples. In certain embodiments, successive serum samples comprise at least five serum samples. In certain embodiments, successive serum samples comprise at least six serum samples. In certain embodiments, successive serum samples comprise at least seven serum samples.

In certain embodiments, the ALS patient is assessed to be responsive to the ALS therapy if the serum concentration of the immune-based biomarker decreases or remains within the reference concentration in a majority of 3 or more successive serum samples taken from the ALS patient.

It will be understood that, in certain embodiments, when assessing responsiveness of the ALS patient to an ALS therapy by comparing serum concentration of each of one or more immune-based biomarkers to a reference concentration for each of the one or more immune-based biomarkers in successive serum samples, the ALS patient can be assessed to be responsive to the ALS therapy if a majority of the successive serum samples show decrease in concentration relative to a concentration in an initial serum sample, or if a majority of successive serum samples remain with a reference concentration.

The successive serum samples can, for example, be consecutive. In some embodiments, the successive serum samples are non-consecutive.

In certain embodiments, a serum sample is taken the same day on which an ALS therapy is administered to the ALS patient. In certain embodiments, a serum sample is taken 1, 2, 3, 4, 5, 6 or 7 days after the day on which an ALS therapy is administered to the ALS patient.

In certain embodiments, the initial serum sample taken after an ALS therapy is administered is taken the same day on which an ALS therapy is administered to the ALS patient. In certain embodiments, the initial serum sample is a serum sample taken after an ALS therapy is administered is taken 1, 2, 3, 4, 5, 6 or 7 days after the day on which an ALS therapy is administered to the ALS patient.

In certain embodiments, one or more successive serum samples are taken 1, 2, 3, 4, 5, 6 or 7 days, one week, two weeks, three weeks or four weeks apart.

In certain embodiments provided herein is a method for treating an ALS patient comprising administering a first ALS therapy to the ALS patient; assessing the responsiveness of the ALS patient to the first ALS therapy; and (i) continue administering the first ALS therapy to the ALS patient if the ALS patient is assessed to be responsive to the first ALS therapy, or (ii) discontinue administering the first ALS therapy to the ALS patient if the ALS patient is assessed to be non-responsive to the first ALS therapy and administer a second ALS therapy to the patient, wherein the first and second ALS therapies are different ALS therapies; wherein the responsiveness of the ALS patient to the first ALS therapy comprises comparing the serum concentration of an immune-based biomarker to a reference concentration and the ALS patient is assessed to be responsive to the first ALS therapy if the serum concentration of the immune-based biomarker decreases or remains within the reference concentration in successive serum samples taken from the ALS patient, otherwise the ALS patient is assessed to be non-responsive to the first ALS therapy.

In certain embodiments, the first and/or second ALS therapy comprises administering one or more Treg infusions to the patient.

In certain embodiments, the first and/or second ALS therapy comprises administering a CTLA-4 fusion protein and IL-2 to the patient. In some embodiments, the CTLA-4 fusion protein is abatacept. In certain embodiments, the IL-2 is aldesleukin.

In certain embodiments, the first and/or second ALS therapy comprises administering one or more Treg extracellular vesicle (EV), e.g., exosome, infusions to the patient.

In certain embodiments, provided herein is a method for treating ALS in a patient diagnosed therewith, the method comprising: administering a first ALS therapy to the ALS patient; assessing the responsiveness of the ALS patient to the first ALS therapy; and continuing to administer the first ALS therapy to the ALS patient if the ALS patient is assessed to be responsive to the first ALS therapy, wherein the responsiveness of the ALS patient to the ALS therapy comprises comparing the serum concentration of at least one immune-based biomarker to a reference concentration of the of least one immune-based biomarker, wherein the ALS patient is assessed to be responsive to the first ALS therapy if the serum concentration of the immune-based biomarker decreases or remains within the reference concentration in at least one successive serum sample taken from the ALS patient.

In particular embodiments of the above method for treating ALS, in instances where a patient is found to not be responsive to the first ALS therapy, the method further comprises administering to the patient a second ALS therapy. In specific embodiments, the method may further comprise, following the administering the second ALS therapy to the ALS patient, assessing the responsiveness of the ALS patient to the second ALS therapy, and continuing to administer the second ALS therapy to the ALS patient if the ALS patient is assessed to be responsive to the second ALS therapy, wherein the responsiveness of the ALS patient to the ALS therapy comprises comparing the serum concentration of at least one immune-based biomarker to a reference concentration of the of least one immune-based biomarker, wherein the ALS patient is assessed to be responsive to the second ALS therapy if the serum concentration of the immune-based biomarker decreases or remains within the reference concentration in at least one successive serum sample taken from the ALS patient.

In certain embodiments, the first or second ALS therapy comprises administering one or more Treg infusions to the patient.

In certain embodiments, the first or second ALS therapy comprises administering a CTLA-4 fusion protein and IL-2 to the patient. In some embodiments, the CTLA-4 fusion protein is abatacept. In certain embodiments, the IL-2 is aldesleukin.

In certain embodiments, the first or second ALS therapy comprises administering one or more Treg extracellular vesicle (EV), e.g., exosome, infusions to the patient.

In certain embodiments provided herein are methods for treating an ALS patient comprising administering a combination of ALS therapies to the ALS patient; assessing the responsiveness of the ALS patient to the combination of ALS therapies administered; and continue administering the combination of ALS therapies to the ALS patient if the ALS patient is assessed to be responsive to the combination of ALS therapies; wherein the responsiveness of the ALS patient to the combination of ALS therapies comprises comparing the serum concentration of one or more immune-based biomarkers to a reference concentration and the ALS patient is assessed to be responsive to the ALS therapy if the serum concentration of the one or more immune-based biomarker decreases or remains within its respective reference concentration in successive serum samples taken from the ALS patient.

In certain embodiments of the methods provided herein, the responsiveness of the ALS patient to an ALS therapy, or to a combination of ALS therapies, comprises comparing the serum concentration of a plurality of immune-based biomarkers to their reference concentrations, and the ALS patient is assessed to be response to the ALS therapy, or combination of ALS therapies, if the serum concentration of the plurality of immune-based biomarkers decrease or remains within their respective reference concentration in successive serum samples. In some embodiments, the plurality of immune-based biomarkers comprise 2, 3, 4, 5, 6, 7, 8, 9 or 10 biomarkers. Immune-based biomarkers can, for example, be any of the serum immune-based biomarkers described herein.

In yet other embodiments of a method provided herein for treating an ALS patient with an ALS therapy (e.g., Treg therapy), the method further comprises performing an additional therapeutic intervention. An additional therapeutic intervention can, for example, be ventilator use, wheelchair use, breathing care, physical therapy (e.g., to address pain, walking, mobility, low-impact exercises), occupational therapy, speech therapy, percutaneous endoscopic gastrostomy (PEG), and palliative care (e.g., medical management for muscle spasms, hypersalivation, pseudobulbar affect, cognitive impairment, and depression).

In some embodiments, provided herein are methods of treating an ALS patient comprising administering an ALS therapy to the patient, wherein the ALS therapy comprises administering IL-2, e.g., aldesleukin, and CTLA-4 fusion protein, e.g., abatacept, as a combination therapy to the patient, assessing responsiveness of the ALS patient to the ALS therapy, wherein (i) when the concentration of a serum-immune based biomarker decreases over a period of time of 2, 3, 4, 5, 6, 7, 8, 9, 10, 11, 12, or more weeks during which the ALS therapy was administered to the ALS patient, or when the concentration of the serum-immune based biomarker remains elevated within a reference concentration range over a period of time of 2, 3, 4, 5, 6, 7, 8, 9, 10, 11, 12, or more weeks during which the ALS therapy was administered to the ALS patient, the patient is responsive to the ALS therapy; and (ii) when the concentration of the serum-immune based biomarker increases over a period of time of 2, 3, 4, 5, 6, 7, 8, 9, 10, 11, 12, or more weeks during which the ALS therapy was administered to the ALS patient, or when the concentration of the serum-immune based biomarker remains elevated over a reference concentration range over a period of time of 2, 3, 4, 5, 6, 7, 8, 9, 10, 11, 12, or more weeks during which the ALS therapy was administered to the ALS patient, the patient is non-responsive to the ALS therapy; and, optionally, further administering the ALS therapy to the ALS patient where the ALS patient is assessed to be responsive to the ALS therapy. The serum immune-based biomarker can, for example, be any serum immune-based biomarker described herein. In certain embodiments the serum immune-based biomarker is ox-LDL. In certain embodiments the serum immune-based biomarker is 4-HNE.

In some embodiments, provided herein are methods of treating an ALS patient comprising administering an ALS therapy to the patient, wherein the ALS therapy comprises administering a Treg EV, e.g., a Treg exosome composition to the patient, assessing responsiveness of the ALS patient to the ALS therapy, wherein (i) when the concentration of a serum-immune based biomarker decreases over a period of time of 2, 3, 4, 5, 6, 7, 8, 9, 10, 11, 12, or more weeks during which the ALS therapy was administered to the ALS patient, or when the concentration of the serum-immune based biomarker remains elevated within a reference concentration range over a period of time of 2, 3, 4, 5, 6, 7, 8, 9, 10, 11, 12, or more weeks during which the ALS therapy was administered to the ALS patient, the patient is responsive to the ALS therapy; and (ii) when the concentration of the serum-immune based biomarker increases over a period of time of 2, 3, 4, 5, 6, 7, 8, 9, 10, 11, 12, or more weeks during which the ALS therapy was administered to the ALS patient, or when the concentration of the serum-immune based biomarker remains elevated over a reference concentration range over a period of time of 2, 3, 4, 5, 6, 7, 8, 9, 10, 11, 12, or more weeks during which the ALS therapy was administered to the ALS patient, the patient is non-responsive to the ALS therapy; and, optionally, further administering the ALS therapy to the ALS patient where the ALS patient is assessed to be responsive to the ALS therapy. The serum immune-based biomarker can, for example, be any serum immune-based biomarker described herein. In certain embodiments the serum immune-based biomarker is ox-LDL. In certain embodiments the serum immune-based biomarker is 4-HNE.

It is contemplated that, in some embodiments of the preceding aspects and methods described herein, one or more additional biomarkers (such as serum immune-based biomarkers) are employed besides the biomarkers disclosed herein.

### 6. EXAMPLES

### 6.1 Assessment of Biomarker Concentrations

Biomarker concentrations in sera were determined by enzyme-linked immunosorbent assay (ELISA), or using the analysis service (OLINK TARGET 48) performed by OLINK PROTEOMICS (Watertown, MA). Sera was obtained from groups of people including those diagnosed with amyotrophic lateral sclerosis (ALS), healthy volunteers, and people diagnosed with dementia (Alzheimer's Disease), frontotemporal dementia, or Parkinson's Disease. In the following, where a given biomarker concentration was determined in an ALS patient prior to any ALS therapy (“untreated patient”), this is referred to as a “baseline value.”

### 6.2 ALS Therapy: T-Regulatory Cells (Tregs)

Eight patients diagnosed with ALS were administered ALS therapy comprising Treg infusions. Inclusion criteria included, inter alia, informed consent and medical record documentation of a decline in ALSFRS-R total score of at least two points in the 90 days prior to screening or at least four points over the 180 days prior to screening. Treg therapy includes taking T-regulatory cells (Tregs) from the patient (leukapheresis), increasing the cell number in a lab, and returning the Tregs in monthly intravenous (IV) infusions back to the same patient (dose of 1×10⁶ cells/kg), plus three times per week subcutaneous interleukin-2 injections. Biomarker concentration was determined in sera that was typically collected from a patient a day following Treg infusion. Patients' Treg numbers and suppressive function were also assayed.

Patients were closely monitored for adverse effects and changes in disease progression rates. To monitor the responsiveness of the ALS patients to treatment, the revised ALS Functional Rating Scale (ALSFRS) and/or Appel ALS Rating Scale (AALS) (Haverkamp et al., Brain, 118 (Pt 3): 707-19 (1995)), which incorporate muscle strength and dysfunction, activities of daily living and pulmonary function, were performed immediately before each Treg infusion, every 2 weeks during each round of infusions, and monthly after each round.

### 6.3 Biomarker Panel: Certain Biomarkers Correlate to ALS Progression

Sera was drawn from 8 rapidly progressing untreated patients with ALS (“Fast ALS patients”) and 8 slowly progressing untreated patients with ALS (“Slow ALS patients”), and 9 age-matched healthy controls. (“healthy controls”). Biomarker concentrations were determined in the sera for a panel of 48 biomarkers. Several of the biomarkers were not detectable in sera, while detectable levels of 43 biomarkers were observed.

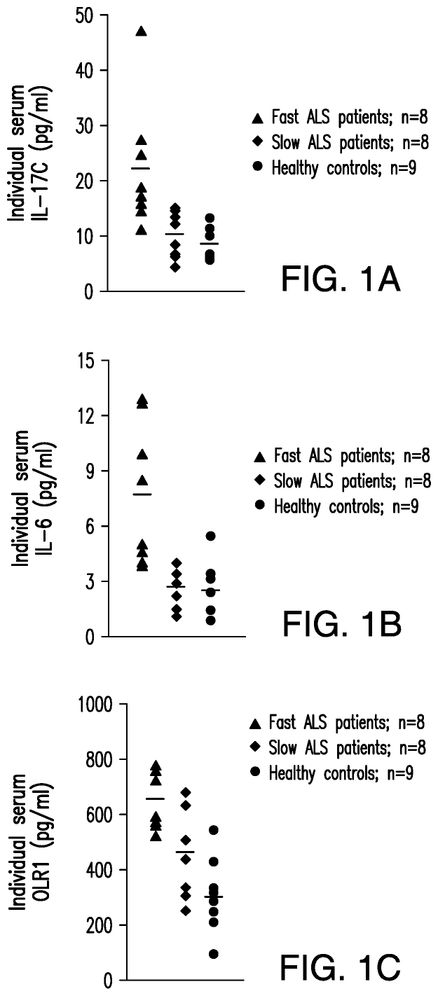

While a number of these biomarkers had concentrations that did not appear to differentiate between the three groups, results provided herein show representative biomarkers where higher biomarker concentrations were observed to correlate with faster progression of ALS, including IL-17C, IL-6 and OLR1, as shown in FIG. 1A (IL-17C), FIG. 1B (IL-6), and FIG. 1C (OLR1) as compared to healthy controls.

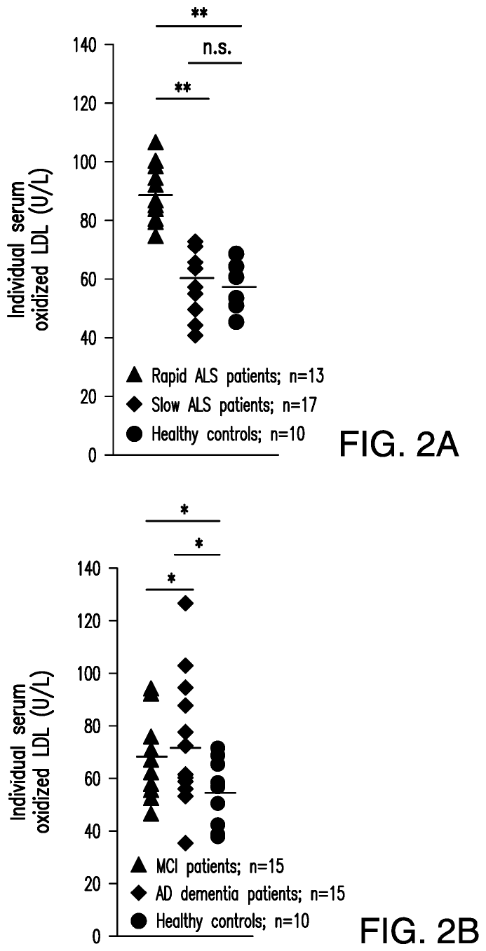

Oxidized LDL (ox-LDL) was also measured in serum samples taken from 13 patients with rapidly progressing ALS, 17 ALS patients with slow progressing ALS and from 10 age-matched controls. As shown in FIG. 2A, ox-LDL was elevated in sera from rapidly progressing patients compared with slowly progressing patients (n=17, p<0.001) or HC (p<0.001) [F (2, 37)=49.78, p<0.001]; ox-LDL was not increased in sera from slowly progressing patients compared with HC (p=0.243). Serum samples were also drawn from patients with Alzheimer's Disease (AD) and with mild cognitive impairment (MCI), and the levels of ox-LDL was compared to that of heathy controls, as shown in FIG. 2B. Comparisons were performed using ANOVA for more than 2 groups or Student's t-test for two groups. The ANOVA is presented with the degrees of freedom, F value, and p value. The Student's t-test is presented with a p value.

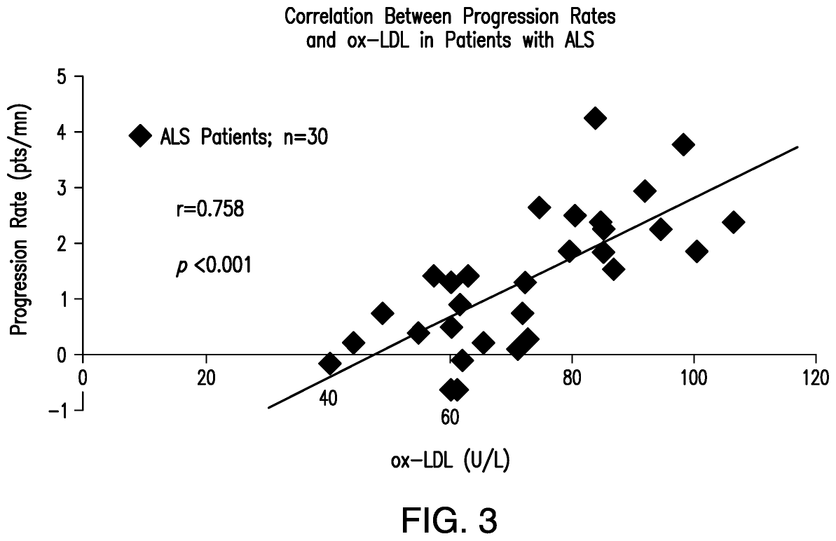

When determining biomarker concentration levels in individual serum samples drawn from untreated ALS patients (including those indicated in the preceding paragraphs and/or from additional studies), biomarkers with serum concentrations in untreated ALS patients that appear to correlate with disease burden, progression rates, and/or survival rates include IL-17C, IL-6, OLR1, ox-LDL, sCD14, LBP, and CRP. An exemplary depiction of results obtained for ox-LDL in a cohort of 30 ALS patients is shown in FIG. 3, demonstrating a correlation of ox-LDL concentration in sera from ALS patients with ALS progression rate as measured in ALSFRS score (points) per month (“pts/mn”).

In FIG. 3, the correlation was analyzed using Spearman Rank Order in SigmaStat software and presented with a rho (r) and p values.

Further, sCD14, LBP and CRP were also tested in sera from patients with Alzheimer's Disease, frontotemporal dementia and Parkinson's Disease and were not found to be elevated.

This example demonstrates that not all biomarkers associated with an inflammatory response have serum concentrations that correlate to ALS, e.g., ALS progression, however, a correlation is present with a number of serum immune-based biomarkers such as, e.g., IL-17C, IL-6, OLR1, ox-LDL, sCD14, LBP, and CRP.

6.4 Administering Treg to ALS Patients: Identifying “Responders” from “Non-Responders”

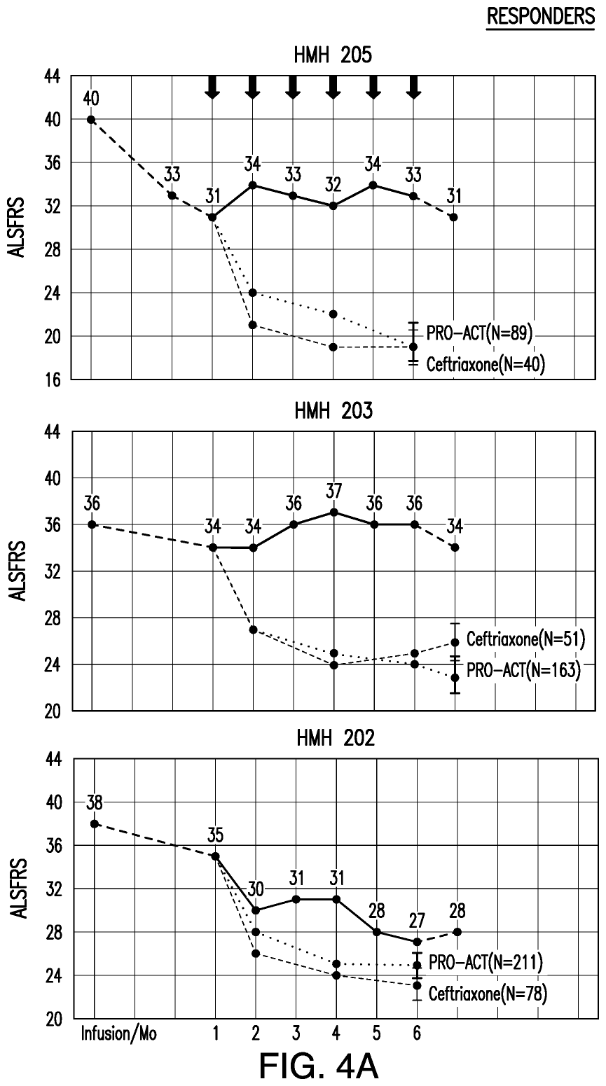

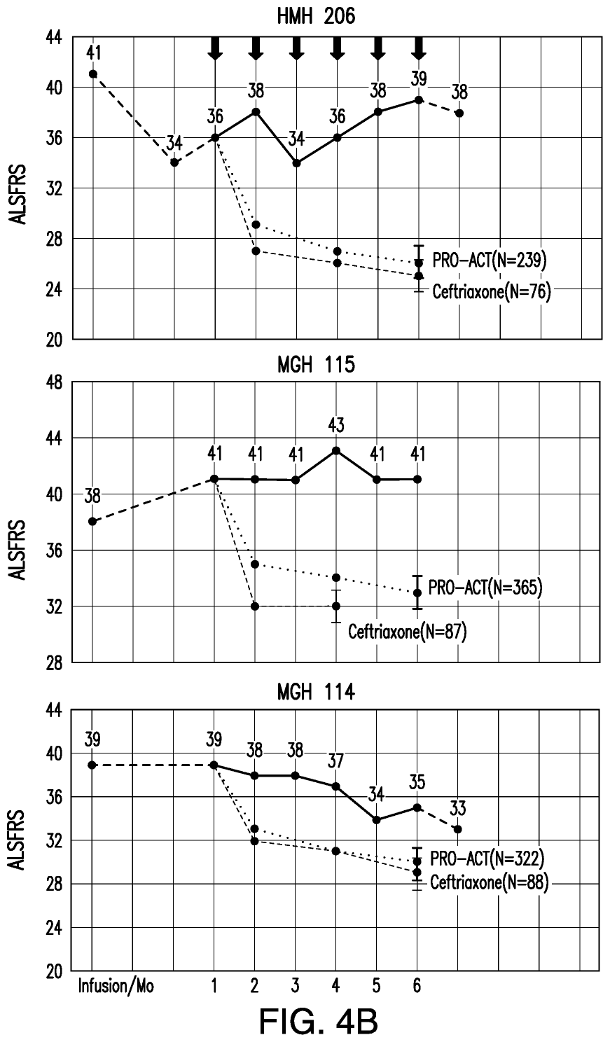

ALS therapy (Treg infusions) was administered in a phase 2a clinical trial to 8 ALS patients (“treated patients”) as described in preceding Example 6.2. ALSFRS scores were determined for each patient prior to, and after onset of TLS therapy. Six patients (subjects nos. 114, 115, 202, 203, 205, and 206) were identified as being responsive (“responders”) to Treg infusions by having ALSFRS scores well above scores in ALS patients administered with ceftriaxone (failed in phase 3 clinical trial when administered to ALS patients, see, e.g., Cudkowicz et al., Lancet Neurol., 13 (11): 1083-1091 (2014)) or to PRO-ACT scores (see FIG. 4A and FIG. 4B).

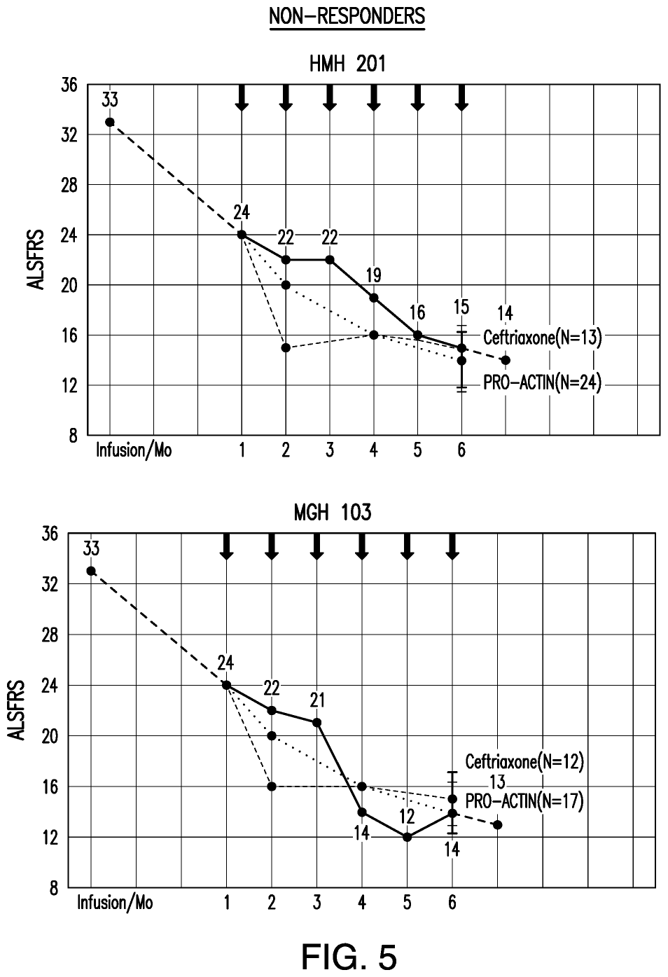

Two patients (subjects 201 and 103) were identified as being non-responsive (“non-responders”) to Treg infusions (see FIG. 5).

### 6.5 ALS Therapy Patient Stratification Using Serum Immune-Based Biomarkers

In this Example immune-based biomarker concentrations in serum samples collected from treated patients prior to Treg infusion and after onset of Treg infusion therapy are reported.

### IL-17F

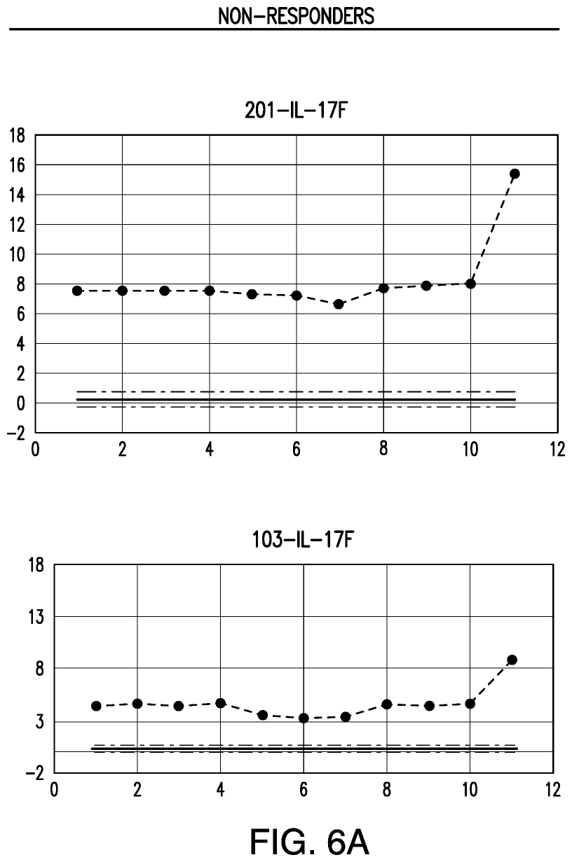

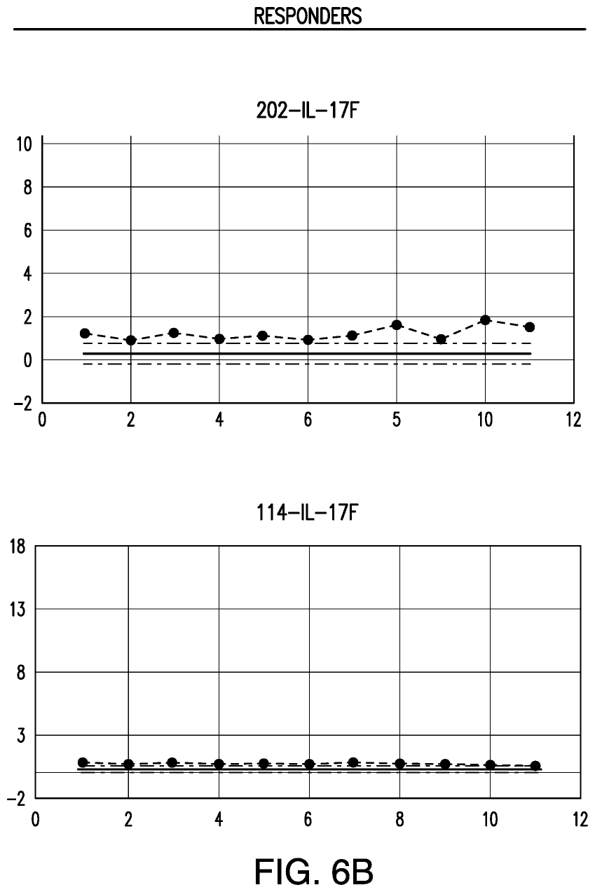

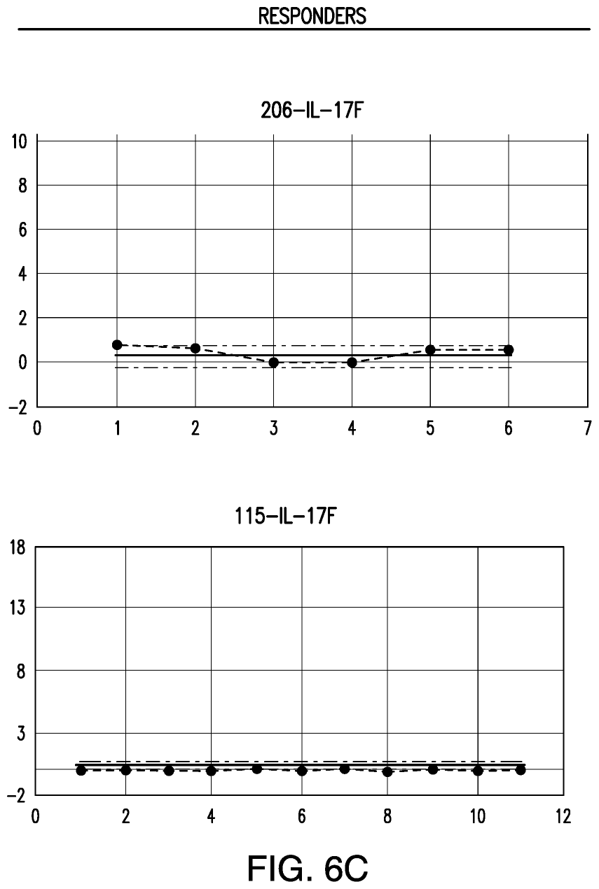

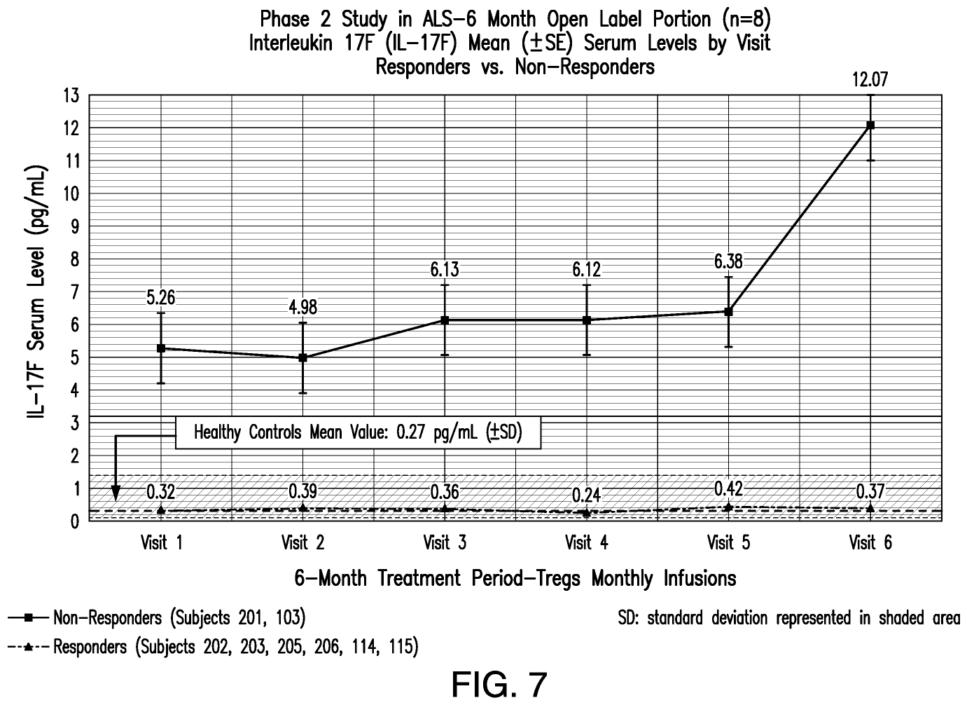

FIG. 6A-6C depicts exemplary graphs of IL-17F serum concentration (pg/mL) in non-responders (FIG. 6A) and responders (FIG. 6B-6C) prior to administration of any Treg infusion (leftmost data point in each graph) and after administration of Treg infusions. Mean average IL-17F serum concentration for healthy controls is depicted in the graphs as a solid straight line in between parallel lines showing one standard deviation above and below the mean average. FIG. 7 depicts the average IL-17F serum concentration for the non-responders and responders, with mean average serum concentration from healthy controls shown as the dashed line.

These results demonstrate that IL-17F serum concentrations in non-responders are elevated as compared to IL-17F concentrations in healthy controls, and as compared to those in responders. The IL-17F serum concentrations are shown to be elevated in non-responders prior to onset of Treg infusion, and remain elevated after Treg infusions.

These results further demonstrate that an elevated serum immune-based biomarker IL-17F concentration is useful to identify whether an ALS patient is likely to be non-responsive to Treg therapy, for example, when the serum sample is collected from the ALS patient prior to onset of Treg therapy, or when collected after a Treg infusion. These results also demonstrate that IL-17F level can indicate efficacy of Treg therapy to a patient, or the responsiveness of a patient to Treg therapy, since its concentration remains elevated in non-responders during administration of a Treg therapy.

### IL-17C

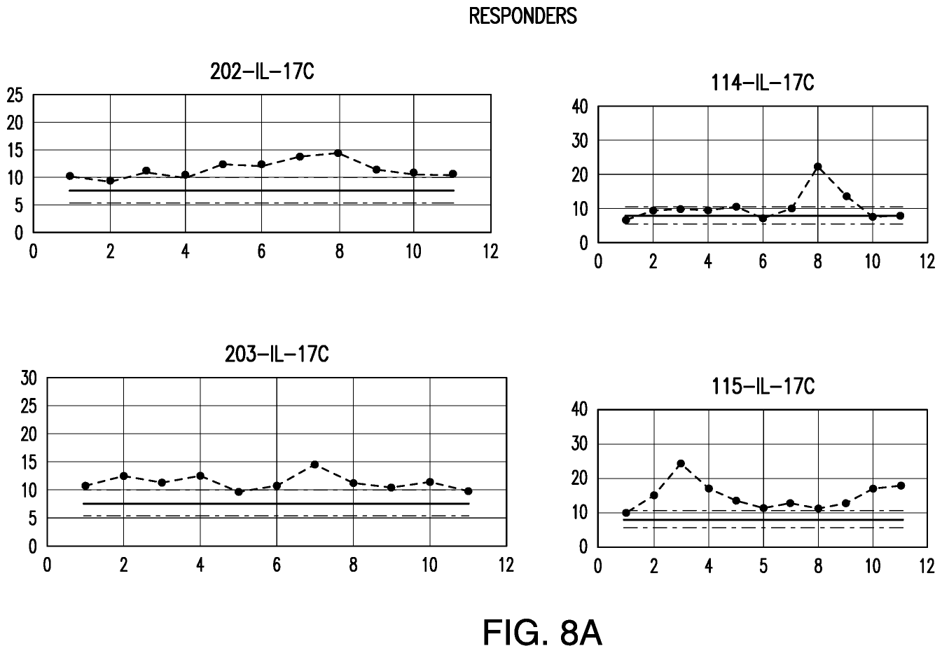

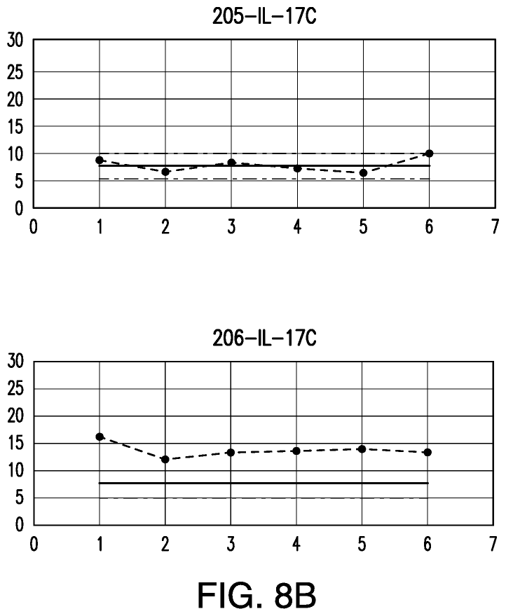

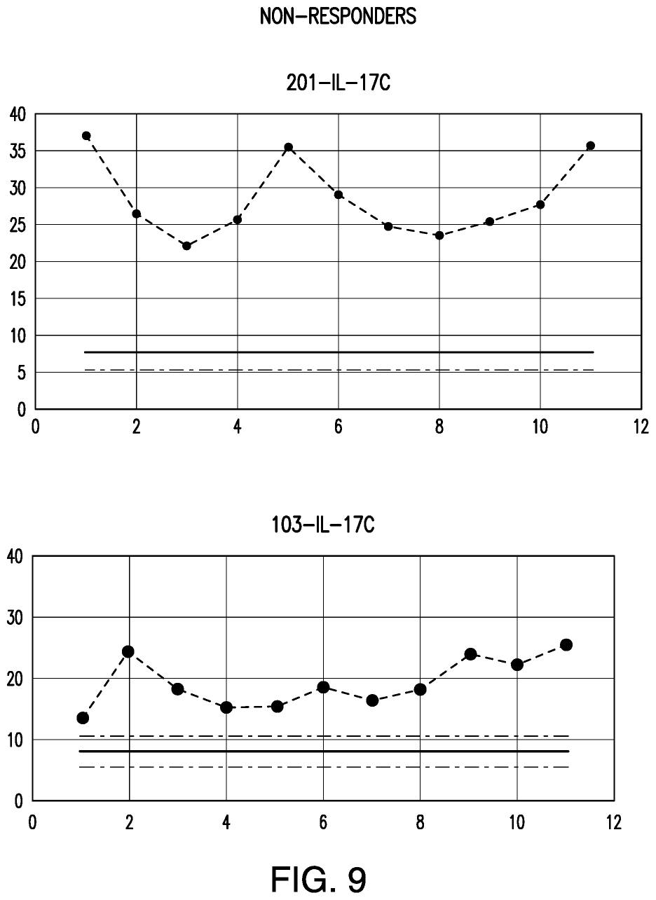

FIG. 8A-8B depicts exemplary graphs of IL-17C serum concentration (pg/mL) in responders prior to administration of any Treg infusion (leftmost data point in each graph) and after administration of Treg infusions. FIG. 9 depicts exemplary graphs of IL-17C serum concentration (pg/mL) in non-responders prior to administration of any Treg infusion (leftmost data point in each graph) and after administration of Treg infusions. In FIG. 8A-8B and FIG. 9, the mean average IL-17C serum concentration for healthy controls is depicted in the graphs as a solid straight line in between parallel lines showing one standard deviation above and below the mean average.

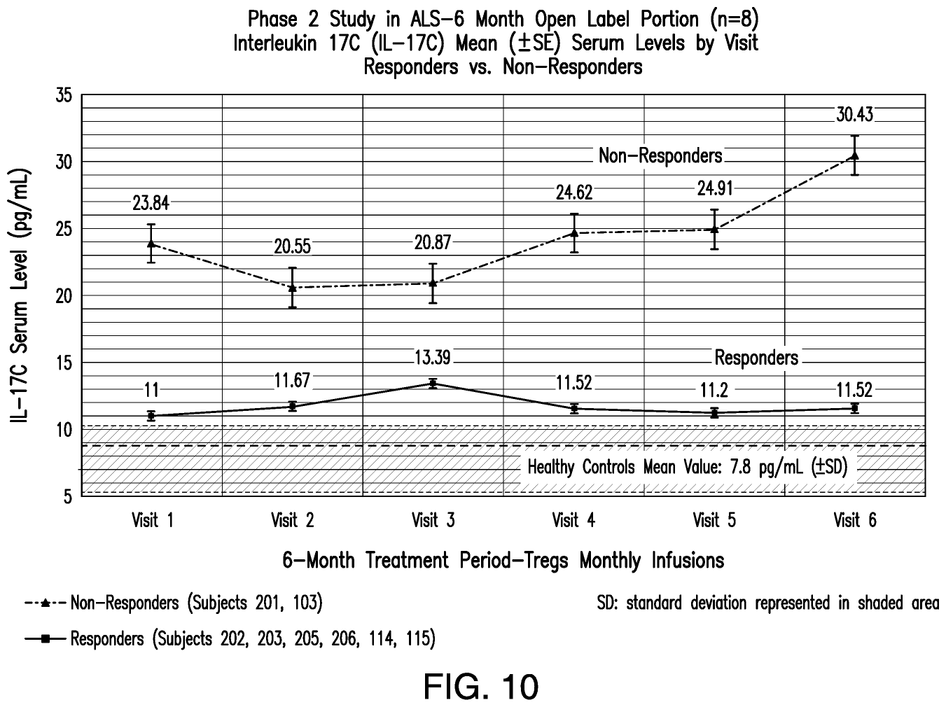

FIG. 10 depicts an exemplary graph of mean average IL-17C serum concentration (pg/mL) in non-responders and responders prior to administration of any Treg infusion (visit 1) and after administration of Treg infusions (visits 2-6), as compared to mean IL-17C serum concentration in healthy controls (shown as a dashed line).

These results demonstrate that IL-17C serum concentrations in non-responders are elevated as compared to IL-17C concentrations in healthy controls, and as compared to the concentrations in responders. The IL-17C serum concentrations are shown to be elevated in non-responders prior to onset of Treg infusion, and remain elevated after Treg infusions.

These results also demonstrate that IL-17C level can indicate efficacy of Treg therapy to a patient, or the responsiveness of a patient to Treg therapy, since its concentration remains elevated in non-responders during administration of a Treg therapy.

### OLR1

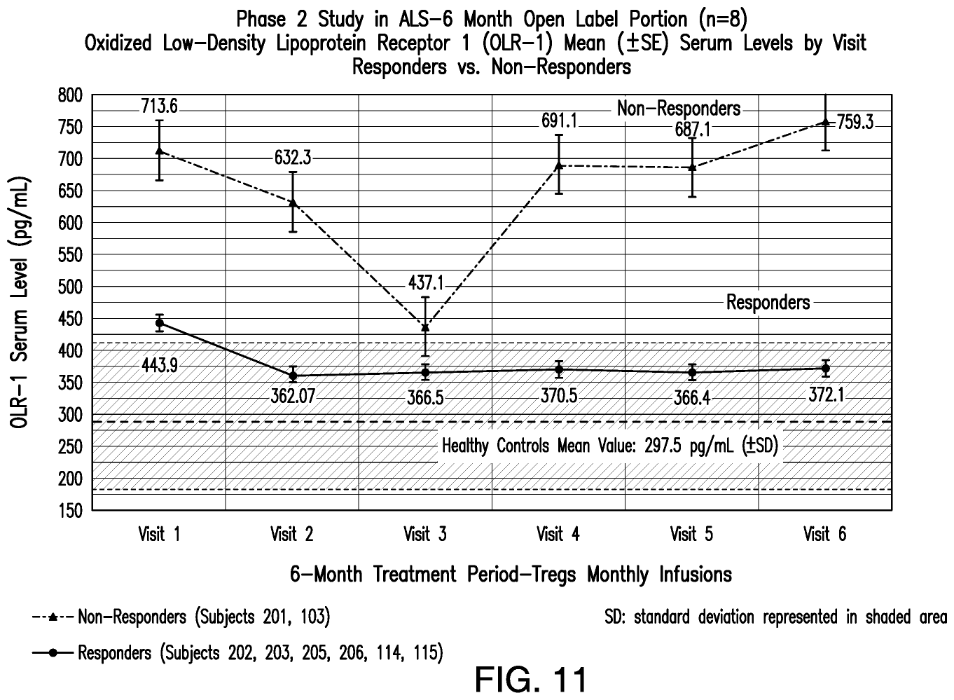

FIG. 11 depicts an exemplary graph of mean average OLR1 serum concentration (pg/mL) in non-responders and responders in serum samples collected on the day of a visit in which a Treg infusion was made (samples were collected on the same day as, but prior to, Treg infusion; visit 1 represents samples collected prior to any Treg infusion). The mean OLR1 serum concentration in healthy controls is shown as a dashed line.

These results demonstrate that OLR1 serum concentrations in non-responders are elevated as compared to OLR1 concentrations in healthy controls, and as compared to concentrations in responders. The OLR1 serum concentrations are shown to remain elevated after Treg infusions in the non-responders.

These results also demonstrate that OLR1 level can indicate efficacy of Treg therapy to a patient, or the responsiveness of a patient to Treg therapy, since its concentration remains elevated in non-responders during administration of a Treg therapy.

### NF-L

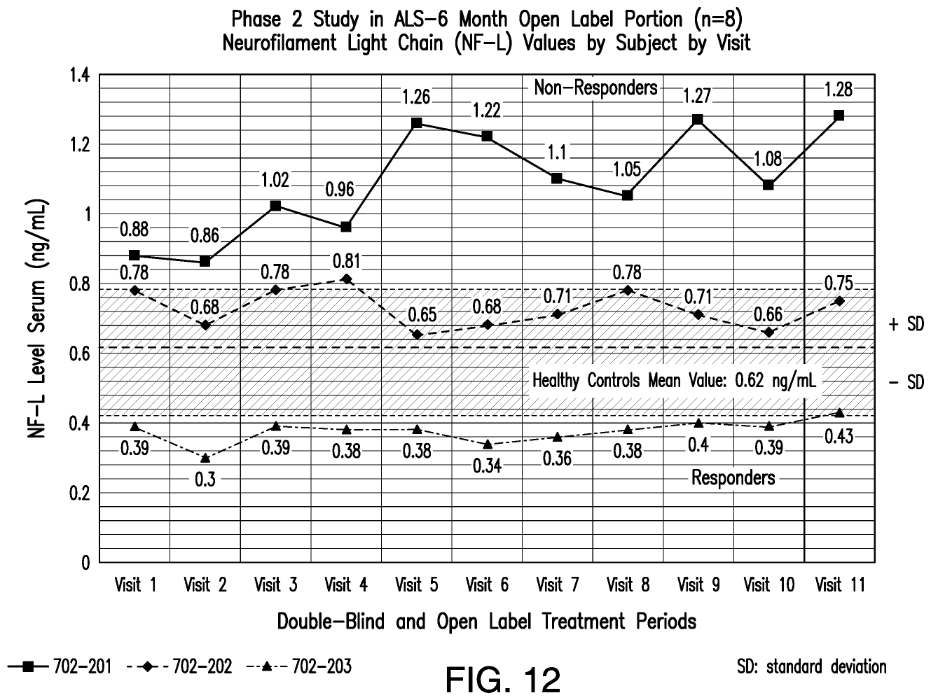

FIG. 12 depicts an exemplary graph of NF-L serum concentration (ng/ml) in a non-responder (subject 201) and two responders (subjects 202 and 203), where serum samples were collected on the day of, but prior to, a Treg infusion (visit 1 represents a serum sample collected prior to any Treg infusion). The mean NF-L serum concentration in healthy controls is shown as dashed line.

These results demonstrate that NF-L serum concentrations in a non-responder are elevated as compared to NF-L concentrations in healthy controls, and as compared to concentrations in responders. The NF-L serum concentration is shown to remain elevated after Treg infusions in the non-responder.

These results also demonstrate that NF-L level can indicate efficacy of Treg therapy to a patient, or the responsiveness of a patient to Treg therapy, since its concentration remains elevated in non-responders during administration of a Treg therapy.

### Ox-LDL

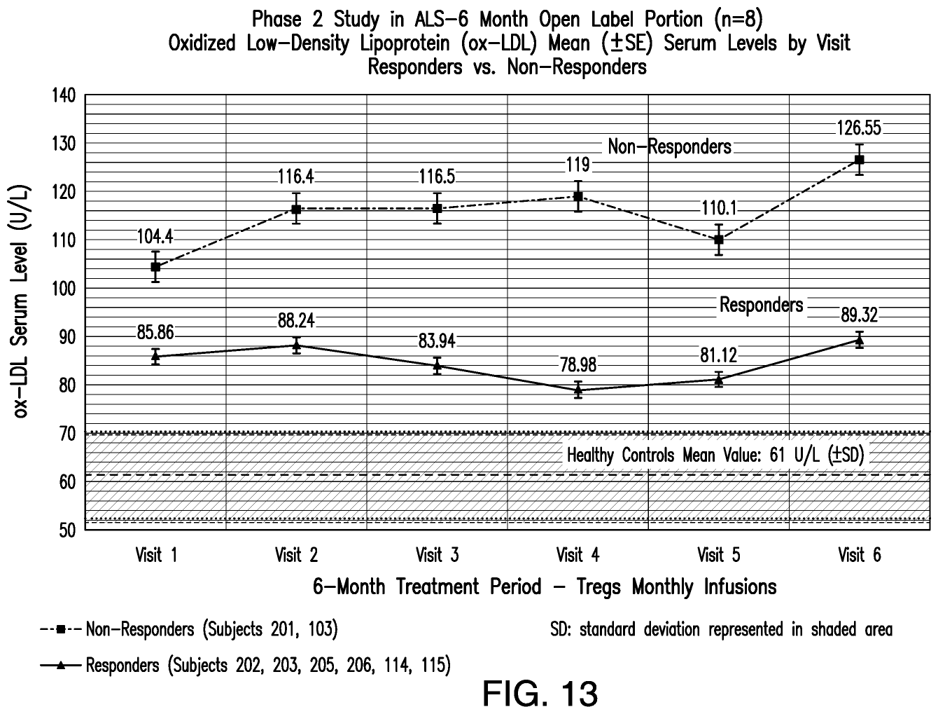

FIG. 13 depicts an exemplary graph of mean average ox-LDL serum concentration (U/L) in non-responders and responders in serum samples collected from subjects on the day of, but prior to, a Treg infusion (visit 1 represents a sample taken prior to any Treg infusion), as compared to mean ox-LDL serum concentration in healthy controls (shown as a dashed line).

These results demonstrate that ox-LDL serum concentrations in non-responders are elevated as compared to ox-LDL concentrations in healthy controls, and as compared to concentrations in responders. The ox-LDL serum concentrations are shown to remain elevated after Treg infusions in non-responders, whether compared to responders or healthy controls.

These results also demonstrate that ox-LDL level can indicate efficacy of Treg therapy to a patient, or the responsiveness of a patient to Treg therapy, since its concentration remains elevated in non-responders during administration of a Treg therapy.

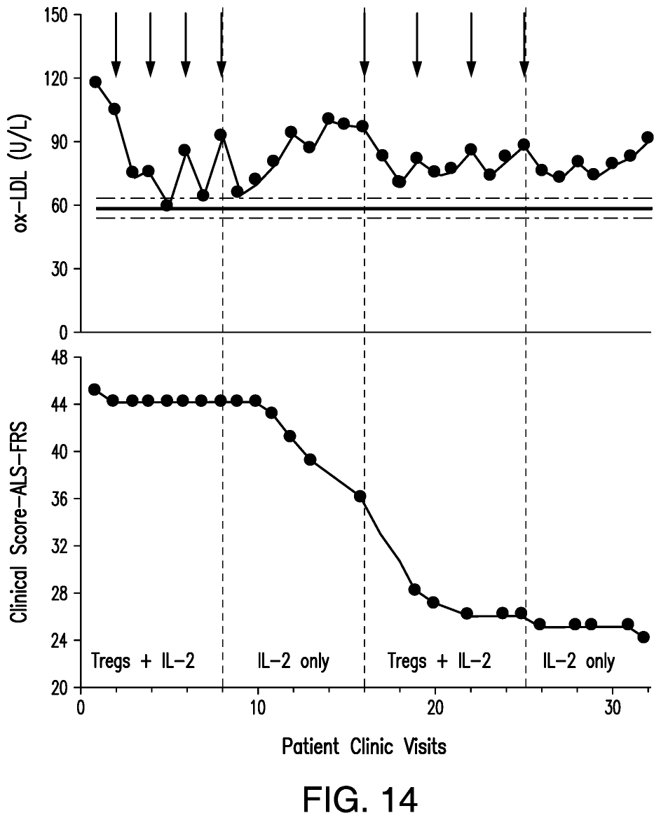

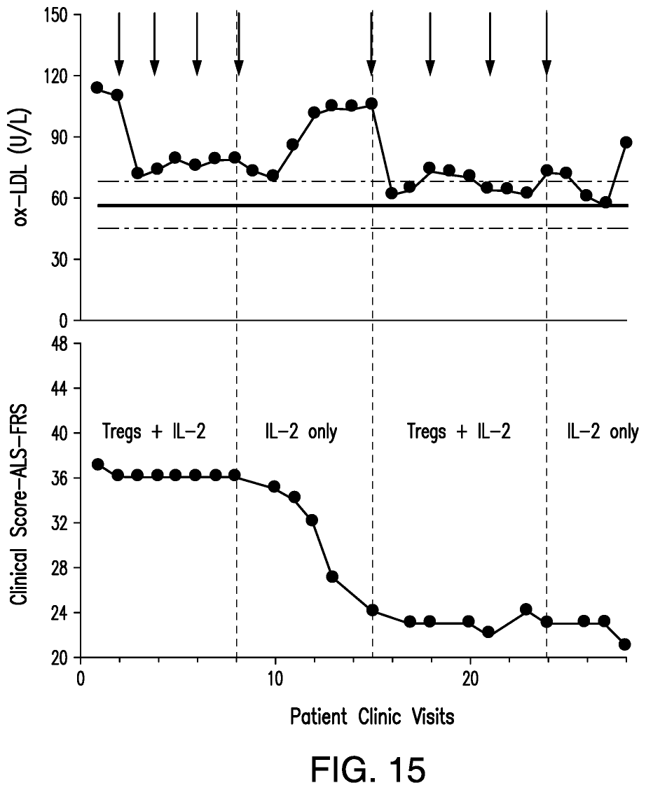

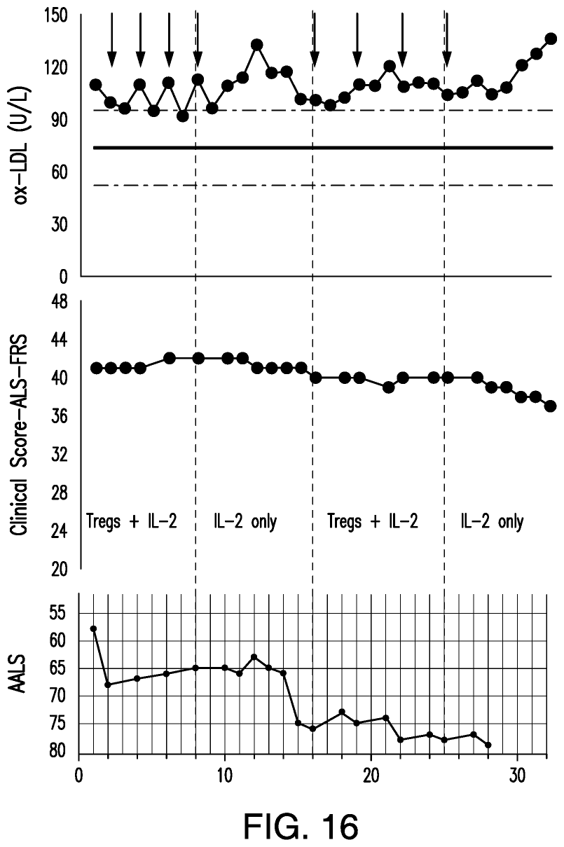

A phase 1 study with 3 patients each receiving Treg infusions plus IL-2 was also conducted. FIG. 14, FIG. 15 and FIG. 16 each depict measurements taken from patients 1, 2 and 3, respectively. The upper panel in each of FIG. 14, FIG. 15, and FIG. 16 indicates the serum concentration of ox-LDL (U/L) prior to and after Treg infusions (the x-axis is a timeline and downward pointing arrows indicate times of Treg infusions). The lower panel in each of FIG. 14 and FIG. 15 depicts the patient's ALSFRS score over the course of the study. The lower panels in FIG. 16 depicts the patient's ALS-FRS and AALS scores over the course of the study. These results demonstrate that Treg infusion results in a decrease in ox-LDL serum concentration, which during a period of no Treg infusions returns to an elevated level. Periods of reduced ox-LDL concentrations following Treg infusions is associated with a plateau in the ALSFRS score (i.e., no decrease), whereas over a period of elevated ox-LDL a decrease in the ALSFRS score was observed. These results provide additional support that elevated levels of ox-LDL can be correlated with progression of ALS.

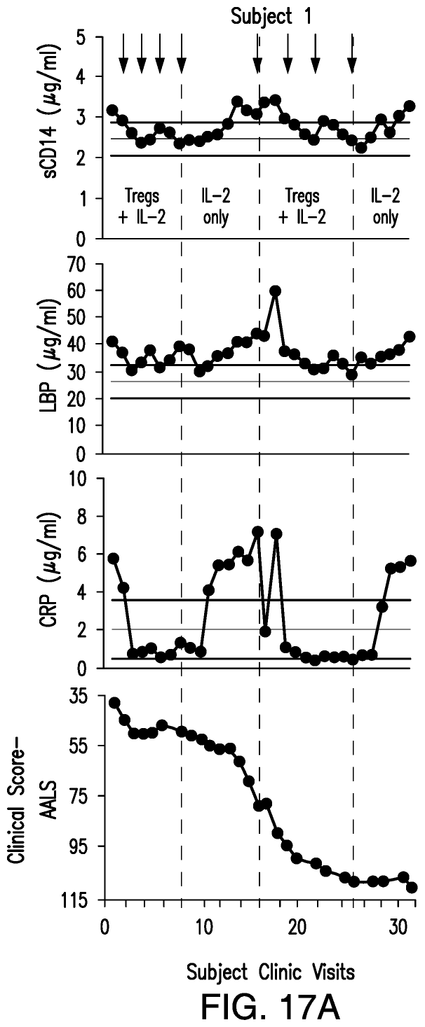

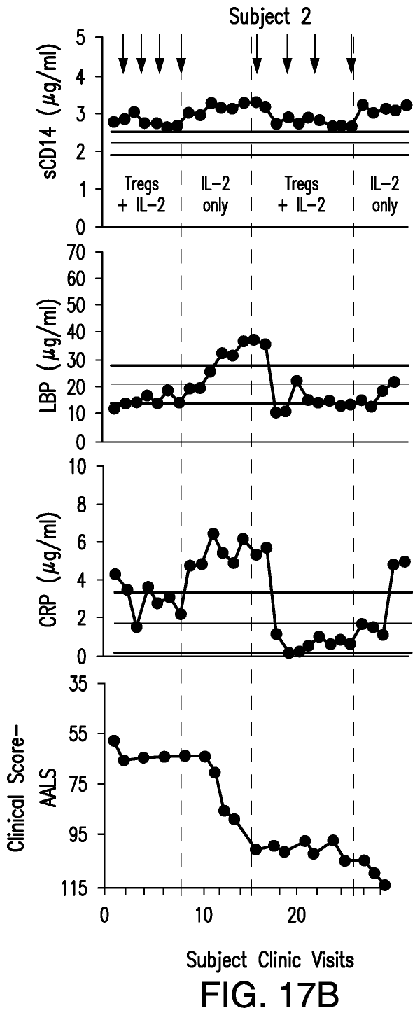

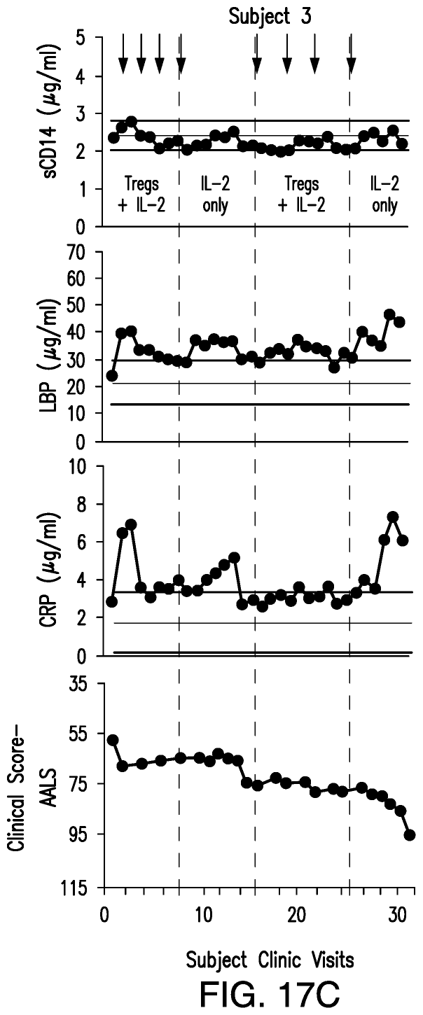

Serum immune-based biomarkers sCD14, LBP and CRP concentrations were also determined in subjects 1, 2 and 3 during Treg infusions as described in the previous paragraph, which are shown, respectively, in FIG. 17A, FIG. 17B and FIG. 17C. In subjects 1 and 2, sCD14, LBP, and CRP fell and rose with Treg+IL-2 treatment (FIG. 17A and FIG. 17B, respectively). sCD14 was relatively unchanged in a slowly progressing subject with ALS and stayed within one standard deviation of the healthy control mean average serum concentration level (FIG. 17C, top panel). LBP and CRP fell and rose with Treg+IL-2 treatment (FIG. 17C, second from top and third from top panels, respectively). In FIGS. 17A to 17C, Arrows indicate Tregs+IL-2 infusion times. IL-2 was administered 3×/week throughout the study. The vertical-dotted lines demarcate Treg+IL-2 therapy or IL-2 only intervals. During the Treg “washout” period, the subjects received IL-2 injections. The three horizontal straight lines show the mean value of each biomarker level in healthy controls (center line), with lines above and below representing+/−one standard deviation of each biomarker level in healthy controls.

The results support that serum immune-based biomarker concentrations correlate with responsiveness to ALS therapies.

### 6.6 Serum Immune-Based Biomarker Concentrations Correlate to Responsiveness to an ALS Therapy

In this Example, immune-based biomarker concentrations in serum samples collected from ALS patients (5 subjects) treated with a combination of IL-2 and abatacept are reported. This Example further demonstrates that serum concentration of certain exemplary immune-based biomarkers correlate with responsiveness to ALS therapy in ALS patients.

Five subjects were enrolled in a phase 1 study evaluating the clinical and biological effects of treatment with abatacept and interleukin-2 (IL-2). Every 2 weeks, subjects received subcutaneous injections of abatacept and IL-2 followed by four additional daily injections of IL-2. Clinic visits occurred every 1 to 2 weeks. Serum samples were collected at each clinic visit, and biomarker concentration in the samples were determined as described below.

### Ox-LDL

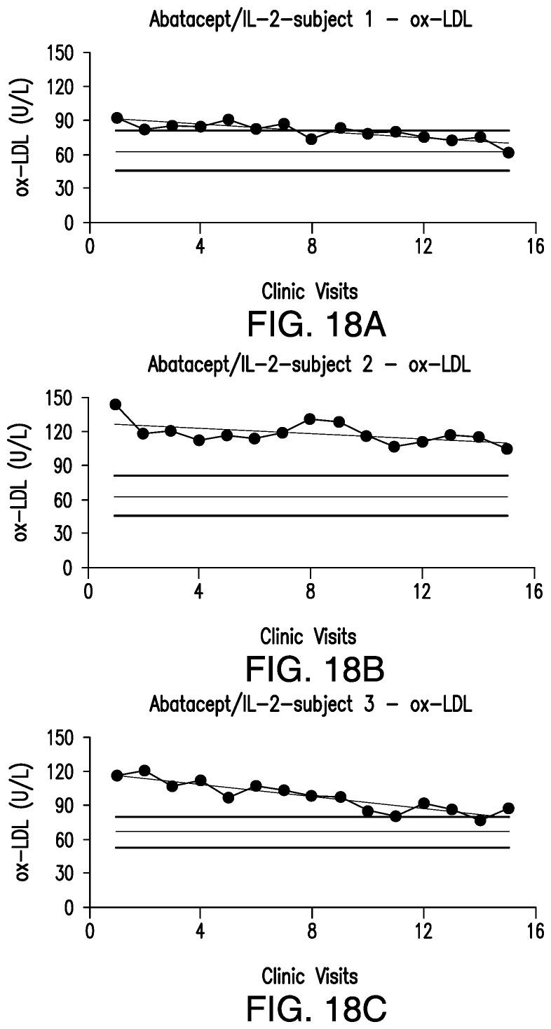

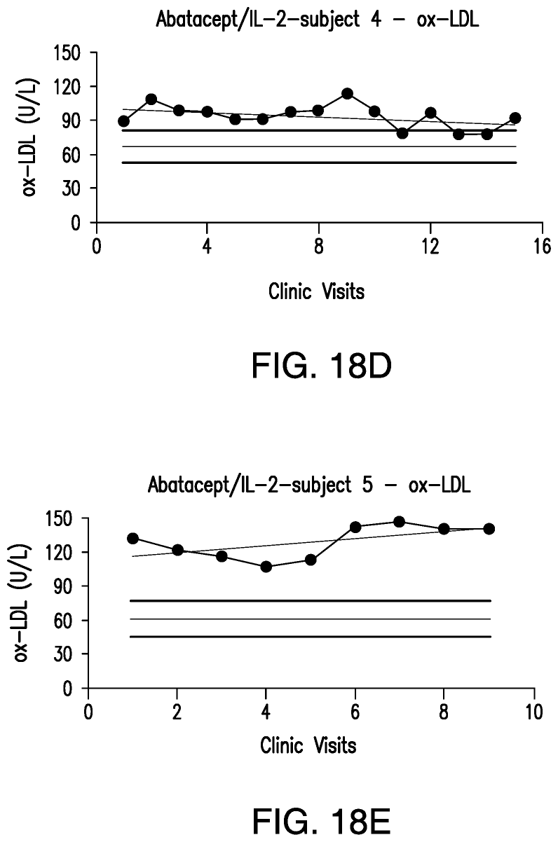

Oxidized-LDL (ox-LDL) levels in the sera were assayed by an ELISA. Subjects 1-5 (FIG. 18A to FIG. 18E) showed variable levels of ox-LDL at baseline. The three horizontal straight lines represent the mean value of ox-LDL levels in healthy controls (center line) and one standard deviation above and below the mean (upper and lower lines, respectively). During the trial, the ox-LDL levels trended downward in subjects 1-4 (FIG. 18A to FIG. 18D) whereas the level trended upward in subject 5 (FIG. 18E). The trajectory of the ox-LDL levels corresponded to the clinical course of each subject. Subjects 1-4 experienced stabilization of their disease progression as ox-LDL levels decreased. Subject 5 experienced rapid clinical progression as the ox-LDL level increased.

### 4-HNE

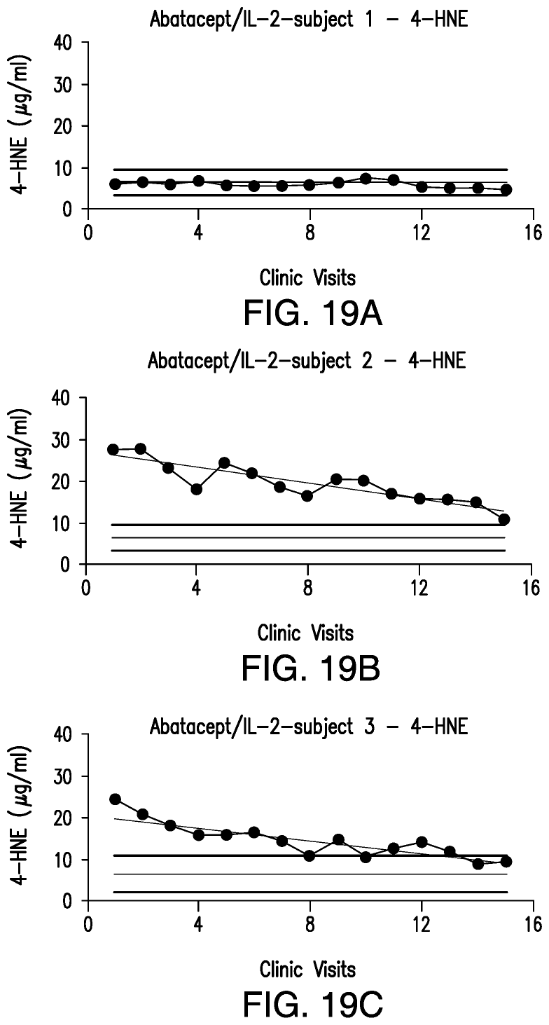

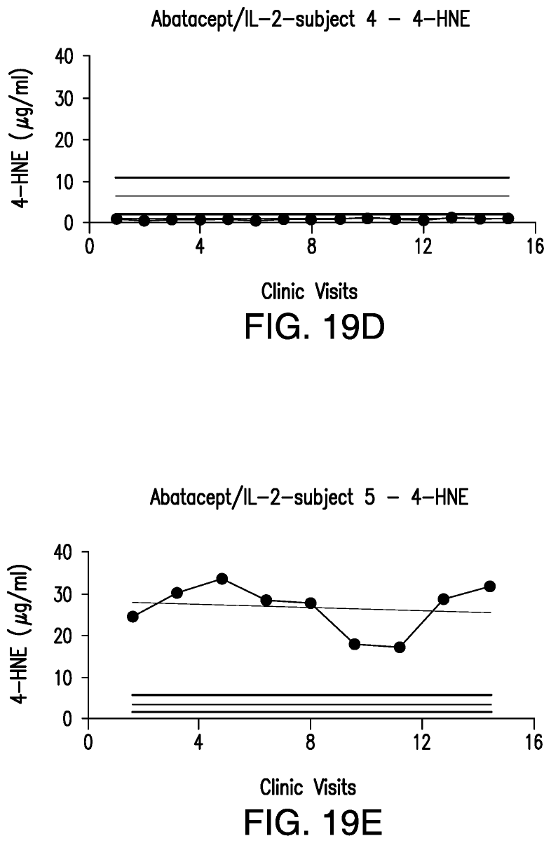

Levels of 4-hydroxynonenal (4-HNE) in the sera were assayed by an ELISA. Subjects 1-5 (FIG. 19A to FIG. 19E) showed variable levels of 4-HNE at baseline. The three horizontal lines represent the mean value of 4-HNE levels in healthy controls (center line) and one standard deviation above and below the mean (upper and lower lines, respectively). During the trial, the 4-HNE levels were within or below the control levels in subjects 1 and 4, trended downward in subjects 2 and 3, and relatively unchanged in subject 5. The levels of 4-HNE corresponded to the clinical course of each subject. Subjects 1˜4 experienced stabilization of their disease progression as 4-HNE levels decreased or were within control levels (FIG. 19A to FIG. 19D). Subject 5 experienced rapid clinical progression as the 4-HNE level remained relatively unchanged (FIG. 19E).

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for selecting a patient for amyotrophic lateral sclerosis (ALS) therapy, the method comprising:
a) determining a concentration of interleukin 17F (IL-17F) in a serum sample collected from a patient diagnosed with or suspected of having ALS, wherein if the IL-17F concentration in the serum sample is at least 2.0 pg/mL the patient is excluded from a treatment with the ALS therapy, otherwise the patient is selected for the treatment with the ALS therapy; and
b) administering the ALS therapy to the selected patient.

2. The method of claim 1, wherein the ALS therapy comprises a T regulatory cell (Treg) infusion.

3. The method of claim 2, wherein the ALS therapy comprises a plurality of Treg infusions.

4. The method of any one of claims 1-3, wherein the serum sample is collected from the patient prior to the ALS therapy.

5. The method of any one of claims 1-3, wherein the serum sample is collected from the patient prior to any Treg infusion.

6. The method of any one of claims 1-3, wherein the serum sample is collected from the patient after a Treg infusion has been administered to the patient.

7. The method of claim 6, wherein the serum sample is collected from the patient on the day following the Treg infusion.

8. A method of treating amyotrophic lateral sclerosis (ALS), the method comprising:
administering an ALS therapy to a patient diagnosed with ALS, wherein it has been determined that a serum sample collected from the patient contains an interleukin 17F (IL-17F) concentration of less than 2.0 pg/mL.

9. The method of claim 8, wherein the ALS therapy comprises a Treg infusion.

10. The method of claim 9, wherein the ALS therapy comprises a plurality of Treg infusions.

11. The method of any one of claims 8-10, wherein the serum sample had been collected from the patient prior to the ALS therapy.

12. The method of any one of claims 8-10, wherein the serum sample had been collected from the patient prior any Treg infusion.

13. The method of any one of claims 8-10, wherein the serum sample had been collected from the patient after a Treg infusion had been administered to the patient.

14. The method of claim 13, wherein the serum sample had been collected from the patient on the day following the Treg infusion.

15. A method for selecting a patient for ALS therapy, the method comprising:
a) determining whether a concentration of at least one serum immune-based biomarker in a serum sample collected from a patient diagnosed with or suspected of having ALS is less than, equal to, or greater than, a reference concentration,
wherein the at least one serum immune-based biomarker is IL-17F, oxidized low-density lipoprotein receptor 1 (OLR1), neurofilament light chain (NF-L), oxidized low-density lipoprotein (ox-LDL), or interleukin 17C (IL-17C); and
wherein if the concentration of the at least one serum immune-based biomarker is greater than the reference concentration, the patient is excluded from a treatment with the ALS therapy, otherwise the patient is selected for the treatment with the ALS therapy; and
b) administering the ALS therapy to the selected patient.

16. The method of claim 15, wherein the concentration of one serum immune-based biomarker is determined.

17. The method of claim 15, wherein concentrations of at least two serum immune-based biomarkers are each determined to be less than, equal to, or greater than, a reference concentration, and if the concentrations of two serum immune-based biomarkers are each determined to be greater than its reference concentration, the patient is excluded from a treatment with the ALS therapy, otherwise the patient is selected for the treatment with the ALS therapy.

18. The method of claim 15, wherein concentrations of at least three serum immune-based biomarkers are each determined to be less than, equal to, or greater than, a reference concentration, and if the concentrations of three serum immune-based biomarkers are each determined to be greater than its reference concentration, the patient is excluded from a treatment with the ALS therapy, otherwise the patient is selected for the treatment with the ALS therapy.

19. The method of claim 15, wherein concentrations of at least four serum immune-based biomarkers are each determined to be less than, equal to, or greater than, a reference concentration, and if the concentrations of four serum immune-based biomarkers are each determined to be greater than its reference concentration, the patient is excluded from a treatment with the ALS therapy, otherwise the patient is selected for the treatment with the ALS therapy.

20. The method of claim 15, wherein concentrations of at least five serum immune-based biomarkers are each determined to be less than, equal to, or greater than, a reference concentration, and if the concentrations of five serum immune-based biomarkers are each determined to be greater than its reference concentration, the patient is excluded from a treatment with the ALS therapy, otherwise the patient is selected for the treatment with the ALS therapy.

21. The method of any one of claims 15-20, wherein the ALS therapy comprises a Treg infusion.

22. The method of claim 21, wherein the ALS therapy comprises a plurality of Treg infusions.

23. The method of any one of claims 15-20, wherein the serum sample is collected from the patient prior to the ALS therapy.

24. The method of any one of claims 15-20, wherein the serum sample is collected from the patient prior to any Treg infusion.

25. The method of any one of claims 15-20, wherein the serum sample is collected from the patient after a Treg infusion has been administered to the patient.

26. The method of claim 25, wherein the serum sample is collected from the patient on the day following the Treg infusion.

27. A method of treating amyotrophic lateral sclerosis (ALS), the method comprising:
administering an ALS therapy to a patient diagnosed with ALS, wherein it has been determined that a serum sample collected from the patient contains a concentration of at least one serum immune-based biomarker that is less than, or equal to, a reference concentration, wherein the at least one serum immune-based biomarker is IL-17F, OLR1, NF-L, ox-LDL, or IL-17C.

28. The method of claim 27, wherein a concentration of one serum immune-based biomarker has been determined to be less than, or equal to, a reference concentration.

29. The method of claim 27, wherein a concentration of at least two serum immune-based biomarkers had been determined to be less than, or equal to, a reference concentration.

30. The method of claim 27, wherein a concentration of at least three serum immune-based biomarkers had been determined to be less than, or equal to, a reference concentration.

31. The method of claim 27, wherein a concentration of at least four serum immune-based biomarkers had been determined to be less than, or equal to, a reference concentration.

32. The method of claim 27, wherein a concentration of at least five serum immune-based biomarkers had been determined to be less than, or equal to, a reference concentration.

33. The method of any one of claims 27-32, wherein the ALS therapy comprises a Treg infusion.

34. The method of claim 33, wherein the ALS therapy comprises a plurality of Treg infusions.

35. The method of any one of claims 27-32, wherein the serum sample had been collected from the patient prior to the ALS therapy.

36. The method of any one of claims 27-32, wherein the serum sample had been collected from the patient prior any Treg infusion.

37. The method of any one of claims 27-32, wherein the serum sample had been collected from the patient after a Treg infusion had been administered to the patient.

38. The method of claim 37, wherein the serum sample had been collected from the patient on the day following the Treg infusion.

39. The method of any one of claims 15-38, wherein the reference concentration is obtained from healthy individuals.

40. The method of any one of claims 15-38, wherein the reference concentration is a concentration in a serum sample obtained from the patient prior to any Treg infusion being administered to the patient.

41. The method of any one of claims 15-38, wherein the reference concentration for each of ox-LDL, OLR1, NF-L, IL-17F, and IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| NF-L | 0.88 ± 0.21 | ng/ml |
| IL-17F | 0.34 ± 0.54 | pg/mL |
| IL-17C | 8.47 ± 3.08 | pg/mL |

42. The method of any one of claims 15-41, wherein the at least one serum immune-based biomarkers includes ox-LDL.

43. The method of any one of claims 15-41, wherein the at least one serum immune-based biomarkers includes OLR1.

44. The method of any one of claims 15-41, wherein the at least one serum immune-based biomarkers includes NF-L.

45. The method of any one of claims 15-41, wherein the at least one serum immune-based biomarkers includes IL-17F.

46. The method of any one of claims 15-41, wherein the at least one serum immune-based biomarkers includes IL-17C.

47. A method of monitoring efficacy of Treg therapy to treat ALS, the method comprising:
a) administering a Treg therapy to a patient diagnosed with ALS, wherein the Treg therapy comprises a first Treg infusion;
b) determining whether a concentration of at least one serum immune-based biomarker in a serum sample collected from the patient after the first Treg infusion is less than, equal to, or greater than, a reference concentration, wherein the at least one serum immune-based biomarker comprises:
oxidized low density lipoprotein (ox-LDL),
oxidized low density lipoprotein receptor 1 (OLR1),
soluble CD14 (sCD14),
lipopolysaccharide binding protein (LBP),
C reactive protein (CRP),
IL-17F, or
IL-17C;
wherein the ALS therapy has poor efficacy where the concentration of the at least one serum immune-based biomarker is greater than its reference concentration.

48. The method of claim 47, further comprising administering the Treg therapy comprising a second Treg infusion to the patient after steps (a) and (b) if the concentration of the at least one serum immune-based biomarker is equal to or less than its reference concentration in step (b).

49. The method of claim 48, wherein Treg therapy comprising a plurality of Treg infusions is administered to the patient if the concentration of the at least one serum immune-based biomarker is equal to or less than its reference concentration in a serum sample collected from the patient after each Treg infusion of the plurality of Treg infusions.

50. The method of any one of claims 47-49, wherein the ALS therapy has poor efficacy where the concentration of one serum immune-based biomarker is greater than its reference concentration.

51. The method of any one of claims 47-49, wherein the ALS therapy has poor efficacy where the concentration of at least 2, at least 3, at least 4, or at least 5 serum immune-based biomarkers are each greater than its reference concentration.

52. A method of treating amyotrophic lateral sclerosis (ALS) comprising:
a) administering a first Treg infusion to a patient diagnosed with ALS;
b) comparing a concentration of at least one serum immune-based biomarker in a serum sample obtained from the patient after the first Treg infusion to a reference concentration;
wherein the at least one immune-based biomarker comprises ox-LDL, OLR1, sCD14, LBP, CRP, IL-17F, or IL-17C; and
c) administering an ALS therapy comprising a second Treg infusion to the patient if the concentration of the at least one serum immune-based biomarker is equal to or below the reference concentration.

53. The method of claim 52 wherein the ALS therapy is administered to the patient if the concentration of one serum immune-based biomarker is equal to or below the reference concentration.

54. The method of claim 52, wherein the ALS therapy is administered to the patient if the concentration of at least 2, at least 3, at least 4 or at least 5 serum immune-based biomarkers are each equal to or below its reference concentration.

55. The method of any one of claims 47-54, wherein the reference concentration for the at least one serum immune-based biomarker is obtained from healthy individuals.

56. The method of any one of claims 47-54, wherein the reference concentration for the at least one serum immune-based biomarker is a concentration in a serum sample obtained from the patient prior to any Treg infusion being administered to the patient.

57. The method of any one of claims 47-54, wherein the reference concentration for each of ox-LDL, OLR1, sCD14, LBP, CRP, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| IL-17F | 0.34 ± 0.54 | pg/mL |
| IL-17C | 8.47 ± 3.08 | pg/mL. |

58. A method for treating a patient with a Treg therapy, wherein the patient is suffering from ALS, the method comprising the steps of:
a) administering to the patient a Treg therapy comprising Treg infusions being administered to the patient on different days;
b) comparing concentrations of at least one serum immune-based biomarker in serum samples to a reference concentration, wherein each of the serum sample is obtained from the patient after a Treg infusion,
wherein the at least one serum immune-based biomarker comprises ox-LDL, OLR1, sCD14, LBP, CRP, IL-17F, or IL-17C; and
c) maintaining the patient on the Treg therapy comprising administering Treg infusions following step (b), if the concentration of the at least one serum immune-based biomarker is at, or less than, the reference concentration in at least one of serum samples.

59. The method of claim 58, wherein the patient is maintained on the Treg therapy if the concentration of the at least one serum immune-based biomarker is at, or less than, the reference concentration in all or at least 50% of the serum samples.

60. The method of any one of claims 58-59, wherein the at least one serum immune-based biomarker consists of one serum immune-based biomarker.

61. The method of any one of claims 58-59, wherein the at least one serum immune-based biomarker comprises at least 2, at least 3, at least 4 or at least 5 serum immune-based biomarkers.

62. The method of any one of claims 58-61, wherein the reference concentration for the at least one serum immune-based biomarker is obtained from healthy individuals.

63. The method of any one of claims 58-61, wherein the reference concentration for the at least one serum immune-based biomarker is a concentration in a serum sample obtained from the patient prior to any Treg infusion being administered to the patient.

64. The method of any one of claims 58-61, wherein the reference concentration for each of ox-LDL, OLR1, sCD14, LBP, CRP, IL-17F, or IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| IL-17F | 0.34 ± 0.54 | pg/mL |
| IL-17C | 8.47 ± 3.08 | pg/mL. |

65. A method for treating ALS in a patient diagnosed therewith, the method comprising:
a) determining whether the concentration of at least one serum immune-based biomarker in a serum sample obtained from a patient diagnosed with ALS is (i) elevated, or (ii) at, or below, a reference concentration, wherein the serum sample is obtained from the patient after being administered with a Treg infusion,
wherein the at least one serum immune-based biomarker comprises ox-LDL, OLR1, sCD14, LBP, CRP, IL-17F, or IL-17C; and
b) administering to the patient a Treg therapy comprising a plurality of Treg infusions if the concentration of the at least one serum immune-based biomarker is determined to be (ii) at, or below, its reference concentration.

66. The method of claim 65, wherein the Treg therapy is administered to the patient if the concentration of one serum immune-based biomarker is determined to be (ii) at, or below, its reference concentration.

67. The method of claim 65, wherein the Treg therapy is administered to the patient if the concentration of at least 2, at least 3, at least 4 or at least 5 serum immune-based biomarkers are each determined to be (ii) at, or below, its reference concentration.

68. The method of any one of claims 65-67, wherein the reference concentration for the at least one serum immune-based biomarker is obtained from healthy individuals.

69. The method of any one of claims 65-67, wherein the reference concentration for the at least one serum immune-based biomarker is a concentration in a serum sample obtained from the patient prior to any Treg infusion being administered to the patient.

70. The method of any one claims 65-67, wherein the reference concentration for each of ox-LDL, OLR1, sCD14, LBP, CRP, IL-17F, and IL-17C is as follows:

| Biomarker | Ref. Conc. | |
|---|---|---|
| ox-LDL | 56.8 ± 7.0 | U/L |
| OLR1 | 299.6 ± 128.1 | pg/mL |
| sCD14 | 2.56 ± 0.44 | ug/mL |
| LBP | 20.18 ± 7.48 | ug/mL |
| CRP | 1.08 ± 1.04 | ug/mL |
| IL-17F | 0.34 ± 0.54 | pg/mL |
| IL-17C | 8.47 ± 3.08 | pg/mL. |

71. The method of any one of claims 47-70, wherein the at least one serum immune-based biomarker comprises ox-LDL, OLR1, sCD14, or LBP.

72. The method of any one of claims 47-70, wherein the at least one serum immune-based biomarker comprises ox-LDL, OLR1, IL-17C, or IL-17F.

73. The method according to any one of claims 47-72, wherein the at least one serum immune-based biomarker comprises ox-LDL.

74. The method according to any one of claims 47-72, wherein the at least one serum immune-based biomarker comprises OLR1.

75. The method according to any one of claims 47-71, wherein the at least one serum immune-based biomarker comprises sCD14.

76. The method according to any one of claims 47-70, wherein the at least one serum immune-based biomarker comprises IL-17C.

77. The method according to any one of claims 47-70, wherein the at least one serum immune-based biomarkers comprises IL-17F.

78. The method according to any one of claims 1-77, wherein the concentration of the at least one serum immune-based biomarker is determined using an enzyme-linked immunosorbent assay (ELISA).

79. A method for treating ALS in a patient diagnosed therewith, the method comprising:
a) determining whether the concentration of at least one serum immune-based biomarker in a serum sample obtained from a patient diagnosed with ALS is at or below that of a reference concentration of the at least one serum immune-based biomarker, wherein the serum sample is obtained from the patient after being administered with a first ALS therapy,
wherein the at least one serum immune-based biomarker comprises ox-LDL, OLR1, sCD14, LBP, CRP, 4-HNE, IL-17F, or IL-17C; and
b) administering to the patient a second ALS therapy if the concentration of the at least one serum immune-based biomarker is determined to be at or below its reference concentration.

80. The method of claim 79, wherein the first ALS therapy is the same as the second ALS therapy.

81. The method of claim 79, wherein the first ALS therapy is different than the second ALS therapy.

82. The method of claim 79, wherein the first and/or second ALS therapy comprises administering one or more Treg infusions to the patient.

83. The method of claim 79, wherein the first and/or second ALS therapy comprises administering a CTLA-4 fusion protein and IL-2 to the patient.

84. The method of claim 83, wherein the CTLA-4 fusion protein is abatacept.

85. The method of claim 79, wherein the first and/or second ALS therapy comprises administering one or more Treg extracellular vesicle (EV), e.g., exosome, infusions to the patient.

86. The method of claim 79, wherein the at least one serum immune-based biomarker comprises ox-LDL or 4-HNE.

87. A method for treating ALS in a patient diagnosed therewith, the method comprising:
administering an ALS therapy to the ALS patient;
assessing the responsiveness of the ALS patient to the ALS therapy; and
continuing to administer the ALS therapy to the ALS patient if the ALS patient is assessed to be responsive to the ALS therapy,
wherein the responsiveness of the ALS patient to the ALS therapy comprises comparing the serum concentration of at least one immune-based biomarker to a reference concentration of the of least one immune-based biomarker, and wherein the ALS patient is assessed to be responsive to the ALS therapy if the serum concentration of the immune-based biomarker decreases or remains within the reference concentration in at least one successive serum sample taken from the ALS patient.

88. The method of claim 87, wherein the responsiveness of the ALS patient to the ALS therapy comprises comparing the serum concentration of an immune-based biomarker to a reference concentration for a set of immune-based biomarkers, and the ALS patient is assessed to be responsive to the ALS therapy if the serum concentration of at least 50% of the immune-based biomarkers in the set decreases or remains within its reference concentration in at least one successive serum sample taken from the ALS patient.

89. The method of claim 88, wherein the set comprises 1, 2, 3, 4, 5, 6, 7, 8, 9, or 10 immune-based biomarkers.

90. The method of claim 87, wherein the ALS patient is assessed to be responsive to the ALS therapy if the serum concentration of the immune-based biomarker decreases or remains within the reference concentration in at least 2, 3, 4, 5, 6, 7, 8, or 9 successive serum samples taken from the ALS patient.

91. The method of claim 87, wherein the ALS patient is assessed to be responsive to the ALS therapy if the serum concentration of the immune-based biomarker decreases or remains within the reference concentration in a majority of 3 or more successive serum samples taken from the ALS patient.

92. The method of any one of claims 87 to 91, wherein the immune-based biomarker comprises ox-LDL, OLR1, sCD14, LBP, CRP, 4-HNE, IL-17F, or IL-17C.

93. The method of any one of claims 87 to 92, wherein the ALS therapy comprises administering one or more Treg infusions to the patient.

94. The method of any one of claims 87 to 92, wherein the ALS therapy comprises administering a CTLA-4 fusion protein and IL-2 to the patient.

95. The method of claim 94, wherein the CTLA-4 fusion protein is abatacept.

96. The method of any one of claims 87 to 92, wherein the ALS therapy comprises administering one or more Treg extracellular vesicle (EV), e.g., exosome, infusions to the patient.
